(19) United States
(12) Patent Application Publication
(10) Pub. No.: US 2026/0125718 A1
(43) Pub. Date: May 7, 2026

(54) GENETICALLY MODIFIED STRAIN OF AUREOBASIDIUM PULLULANS AND USE THEREOF FOR PRODUCING BIOPRODUCT IN HIGH YIELD

(71) Applicant: Safion Renewables Inc., Louisville, CO (US)

(72) Inventors: Lauren Ann Riley Drumm, Lafayette, CO (US); Andrew John Borchert, Arvad, CO (US); Julia Rae Friedman, Boulder, CO (US)

(21) Appl. No.: 19/313,556

(22) Filed: Aug. 28, 2025

Related U.S. Application Data
(60) Provisional application No. 63/689,130, filed on Aug. 30, 2024.

(51) Int. Cl. C12P 7/64; C07K 14/22; C07K 14/39; C12N 15/81

(52) U.S. Cl. CPC C12P 7/64; C07K 14/22; C07K 14/39; C12N 15/815

(57) ABSTRACT

Provided are genetically modified strains of Aureobasidium pullulans that can produce a bioproduct with increased productivity and a method of producing a bioproduct using the same, wherein the genetically modified strain of Aureobasidium pullulans comprises an overexpression of a functional VHb protein and an overexpression of a functional PacC protein and is grown under conditions required to support the production of the bioproduct to collect the bioproduct in a high yield.

### CROSS-REFERENCE TO RELATED APPLICATIONS

The present application claims the benefit of U.S. Provisional Patent Application No. 63/689,130 filed Aug. 30, 2024, which is incorporated herein by reference in its entirety.

### REFERENCE TO A SEQUENCE LISTING XML

The instant application contains a Sequence Listing, which has been submitted electronically in XML format and is hereby incorporated by reference in its entirety. Said XML file, created on Aug. 27, 2025, is named 2025-08-28_Sequence-Listing_19995.0001USU1.xml and is 47,700 bytes in size.

### FIELD OF THE DISCLOSURE

The present disclosure generally relates to methods and compositions of Aureobasidium pullulans strains for producing industrial-levels of renewable bioproducts. Particularly, the present disclosure relates to a genetically modified strains of Aureobasidium pullulans that can produce a bioproduct with increased productivity, yields, and titers, and a method of producing the bioproduct using the same.

### BACKGROUND OF THE DISCLOSURE

Sustainable biotechnology has seen an increase in investment and development as natural resources continue to be depleted and environmentally harmful chemical processes are unsustainable long-term. Microorganisms are often utilized as biocatalysts for manufacturing sustainable products for applications in a variety of industries such as pharmaceuticals, energy, raw materials, pollution prevention, and so forth. Species of a black, yeast-like fungi (belonging to the Aureobasidium genus), have shown promise as scalable, genetically tractable microorganisms with versatile metabolic networks that can be easily modified for producing a plethora of natural products including pullulan, β-glycan, polymalate, melanin, polyol lipids, and aglycone oligo-dihydroxydecanoic acids, which have broad commercial applications within cosmetics, healthcare, chemicals, and fuels industries.

Bioproducts produced by Aureobasidium pullulans strains include mixtures of extracellular polyol lipids (amphiphilic glycolipids comprising a sugar alcohol head group and ester-linked fatty acid tails) and aglycone oligo-dihydroxydecanoic acids (lacking a sugar alcohol head group), or water-insoluble “heavy oils”, that possess renewable applications as antimicrobials, biosurfactants, coconut oil substitutes, and as substrates for low carbon intensity biofuels, cosmetics, flavoring chemicals, and healthcare products. The polyol lipids within heavy oils typically are composed of a mannitol head group bound via ester linkages to between three to five decanoic acid groups. The mannitol headgroup is synthesized from the central metabolite, fructose-6-phosphate via the combined action of mannitol-1-phosphatase and mannitol-1-phosphate dehydrogenase enzymes. The decanoic acid group is created by a highly reduced polyketide synthase (HR-PKS) combining the metabolites acetyl-CoA and malonyl-CoA. Three to five dihydroxydecanoic acid tails can undergo esterification to form the aglycone oligo-dihydroxydecanoic acids. To produce the polyol lipid fraction of heavy oil, a mannitol headgroup and dihydroxydecanoic acid tail are assembled by an esterase enzyme. The heavy oil is secreted from the cell and is capable of spontaneously phase separating from the aqueous growth media.

But complex eukaryotes such as Aureobasidium pullulans are not solely dedicated to producing a single bioproduct, as heavy oil must compete for carbon and energy resources with other biological processes, lowering the potential titers, rates, and yields of heavy oils during fermentation.

Previous methods for optimizing the production of heavy oils have included alterations of growth medium, fermentation conditions, and the introduction of an induction medium. One earlier work related to heavy oil production is described in U.S. Pat. No. 11,352,633, which describes an Aureobasidium pullulans recombinant strain with high-yield heavy oil that was obtained by knocking out a pullulan synthetase gene while overexpressing an ATP-citrate lyase gene. Another work of genetically modifying Aureobasidium pullulans removes a functional polyketide-synthase (PKS4) enzyme for omitting the byproduct melanin from produced bioproducts. This genetic alteration reduces the cost of processing the produced bioproducts such as heavy oils and pullulan, but the production of heavy oils is limited to no more than 22 g/L at maximum titer.

### SUMMARY OF THE DISCLOSURE

Various details of the present disclosure are hereinafter summarized to provide a basic understanding. This summary is not an exhaustive overview of the disclosure and is neither intended to identify certain elements of the disclosure, nor to delineate the scope thereof. Rather, the primary purpose of this summary is to present some concepts of the disclosure in a simplified form prior to the more detailed description that is presented hereinafter.

To improve the heavy oil production on a commercial level, the present disclosure provides a genetically modified strain of Aureobasidium pullulans optimized to produce increased quantities of heavy oils by restructuring host metabolism and gene expression to enhance current heavy oil biosynthesis and secretion. The present disclosure presents genetic changes that divert metabolic resources from irrelevant byproducts and optimize pathways and resources towards extracellular expression of heavy oils.

Accordingly, in one or more aspects, the present disclosure provides a method of producing a bioproduct comprising: growing a genetically modified strain of Aureobasidium pullulans under conditions required to support the production of the bioproduct, wherein the genetically modified strain of Aureobasidium pullulans comprises an overexpression of a functional VHb protein having at least 90% sequence homology with the sequence set forth in SEQ ID NO. 1, and an overexpression of a functional PacC protein; and collecting the bioproduct from the genetically modified strain of Aureobasidium pullulans.

In another aspect, the present disclosure provides a composition comprising a strain of Aureobasidium pullulans, wherein the strain comprises: an overabundance of a functional VHb protein having at least 90% sequence homology with the sequence set forth in SEQ ID NO: 1; and an overabundance of a functional PacC protein, with optionally a loss of a functional creA gene.

Any combinations of the various embodiments and implementations disclosed herein can be used in a further embodiment, consistent with the disclosure. These and other aspects and features can be appreciated from the following description of certain embodiments presented herein in accordance with the disclosure and the accompanying drawings and claims.

### BRIEF DESCRIPTION OF THE DRAWINGS

FIG. 1A-1B depict a representative DNA gene cassette for genetic modification in a strain of Aureobasidium pullulans in an example.

FIG. 2 depicts a non-limiting example of a metabolic pathway genetically modified for enhanced production of heavy oils from a strain of Aureobasidium pullulans.

FIG. 3 is a block diagram of a non-limiting system and method of producing and collecting heavy oils from a modified yeast-like fungus.

FIG. 4 is chart illustrating sucrose consumption in a genetically modified strain having Vhb overexpression in an example.

FIG. 5 is chart illustrating pullulan and oil yields in a genetically modified strain having PacC overexpression in an example.

FIG. 6 is a chart illustrating pullulan and oil titers in a genetically modified strain with pullulanase overexpression in an example.

FIG. 7 is a chart illustrating reduction in pullulan in genetically modified strains having UDPG-pyrophosphorylase knockout in an example.

### DETAILED DESCRIPTION

Discussed herein are a genetically modified strain of Aureobasidium pullulans engineered to produce increased quantities of heavy oils with enhanced productivity, yields, and titers, and associated methodologies. A particular challenge of conventional strains are relatively low yields and the co-expression of irrelevant cellular byproducts, with some paradigms limited to about 22 g/L at maximum titer.

By comparison, the strains discussed herein produce increased desirable bioproducts. For example, in a genetically modified strain, an overexpression of Vitreoscilla hemoglobin (VHb) protein, an overexpression of a functional PacC protein, a loss of functional creA gene, or combinations thereof can help produce the desired increased quantities of heavy oils with preferred productivity, titers, and yields. Such modifications can help divert metabolic resources from competing byproducts, such as pullulan or poly-malic acid (PMA) towards heavy oil production. Various genetic modifications such as knockout of UDPG-pyrophosphorylase genes or overexpression of key regulatory proteins can help produce these results.

In the methods discussed herein, first, production of competing side product, pullulan, is reduced to direct carbon towards heavy oil production and streamline downstream processing. Accordingly, the genes encoding enzymes that catalyze the first step of pullulan production (UDPG-pyrophosphorylase) are knocked out. Further, the gene encoding the enzyme responsible for pullulan degradation (pullulanase) into sugar monomers is overexpressed. The overexpression of this gene allows for conversion of pullulan into heavy oil. Additionally, the heterologous gene encoding Vitreoscilla hemoglobin, vhb, is constitutively expressed to increase heavy oil titers. The production of heavy oil is an oxygen intensive process and overexpression of Vhb increases oxygen availability for the pathway through binding of oxygen. Finally, the native pacC gene is overexpressed to increase heavy oil titers. PacC regulates expression of gene members of the heavy oil production pathway. Overexpression of pacC increases expression of the heavy oil synthesis pathway, increasing heavy oil titers.

The advantages of the strains and methods discussed herein include, but are not limited to, VHb overexpression enabling a 10-30% increase in sucrose consumption rate; PacC overexpression resulting in a 20-70% decrease in pullulan yield and increased oil yields; pullulanase overexpression decreasing final pullulan titers by 20-50% and increasing heavy oil by 10-30%; and UDPG-pyrophosphorylase knockout achieving 20-60% reduction in pullulan production depending on the specific gene sequence targeted. The invention enables production of heavy oils over 30 g/L, representing a significant improvement over existing methods.

Moreover, the advantages of the described strains and methods provide unexpected benefits, including but not limited to benefits to combined modifications, unexpected result from the pullulan pathway disruption, and metabolic flux redirection.

For example, the combination of VHb overexpression with PacC overexpression produced unexpected synergistic effects that would not have been predictable from the individual modifications alone. While VHb overexpression alone demonstrated a 22% increase in sucrose consumption rate, and PacC overexpression alone resulted in a 60% decrease in pullulan production, these modifications work together to redirect metabolic flux toward heavy oil production. In this case, the VHb protein can function as an oxygen-binding protein to enhance oxygen availability for metabolic processes, while PacC serves as a pH-responsive transcription factor regulating the heavy oil biosynthetic pathway. The unexpected result is that combining these disparate mechanisms—oxygen availability enhancement and transcriptional regulation—creates a coordinated metabolic redirection that achieves heavy oil production levels exceeding 30 g/L, representing a significant improvement over the prior art limitation of 22 g/L maximum titer.

In another example, the combination of UDPG-pyrophosphorylase knockout with pullulanase overexpression produced superior results compared to either modification alone. While knockout of individual UDPG-pyrophosphorylase gene sequences achieved 34-58% reduction in pullulan production, and pullulanase overexpression alone decreased pullulan titers by 45%, combining a gene knockout approach with an enzyme overexpression approach created a dual mechanism for pullulan reduction. The result was that the knockout prevents pullulan synthesis at the enzymatic level while the overexpression simultaneously degrades any residual pullulan into sugar monomers that can be redirected toward heavy oil production, creating a more complete metabolic redirection than would be expected from simply adding the individual effects.

In another example, the combination of several successful modifications—VHb overexpression, PacC overexpression, pullulanase overexpression, and UDPG-pyrophosphorylase knockout—produced results in terms of overall metabolic efficiency. While each individual modification addressed different aspects of the metabolic pathway, combining oxygen availability enhancement, transcriptional regulation, competing pathway disruption, and byproduct degradation worked together to create a comprehensive redirection of cellular resources toward heavy oil production. The result is that these modifications worked synergistically to address multiple bottlenecks simultaneously, achieving heavy oil yields that substantially exceed what would be predicted from the sum of individual improvements, demonstrating that the invention solves the technical problem of low heavy oil production through an unexpected, coordinated approach rather than through predictable additive effects.

Definitions. As used herein, the term “Aureobasidium,” “Aureobasidium pullulans,” “A. pullulans,” and grammatical variants thereof, refers generally to a dimorphic black mold fungus comprised of both major yeast cells and minor filamentous cells. Aureobasidium pullulans is a genetically tractable organism with the capability of synthesizing a variety of industrial and pharmaceutical biotechnology byproducts such as pullulan, β-glycan, polymalate (PMA), melanin, gluconic acid, fumaric acid, sideophores, in addition to polyol lipids and aglycone oligo-dihydroxydecanoic acids (heavy oils). Thus, it is an ideal fungus for genetic editing, metabolic engineering, and synthetic biology applications.

As used herein, the term “bioproduct,” and grammatical variants thereof, refers generally to valuable biologically-derived materials and chemicals produced from renewable feedstock.

As used herein, the term “liamocins,” “heavy oils,” “microbial oils,” and grammatical variants thereof, refers generally to molecules within a subclass of biosurfactant glycolipids identified as polyol lipids produced by the Aureobasidium pullulans yeast-like fungus. Liamocins are amphiphilic molecules comprising a polyol head group such as mannitol, arabitol, or glycerol, attached to three to six esterified 3,5-dihydroxydecanoic acid group tails. The 3,5-dihydroxydecanoic acid group tails may be acetylated at the hydroxyl (—OH) group. Heavy oils and microbial oils lacking the polyol head group are classified as aglycone oligo-dihydroxydecanoic acids (DDA) (formerly exophilins).

As used herein, the term “genetically modified,” “cloning,” “transformed,” and grammatical variants thereof, refers generally to streamlined molecular biology techniques to construct and amplify artificial DNA structures such as plasmids or vectors. Artificial DNA structures may be DNA sequences edited in any fashion such as deletion or insertion, joining together in multiple copies of a single gene, or stringing together a mixture of differing genes. Methods may utilize DNA restriction enzyme sites or recombination sites for the excision or insertion of artificial DNA sequences.

As used herein, the term “transformation,” and grammatical variants thereof, refers generally to the genetic manipulation of an organism by transference of exogenous genetic material into the cell of a target organism. This process allows for the transient or permanent introduction into a vulnerable organism, enabling genetic modification.

Dense “heavy oils,” produced by the Aureobasidium pullulans have been characterized by numerous studies as an antiproliferative agent on cancer cells, an antimicrobial agent, and use as a biosurfactant. However, current methods for producing heavy oils with Aureobasidium pullulans are plagued by relatively low yields and the co-expression of irrelevant cellular byproducts.

To address limitations with heavy oil production on a commercial level, the present disclosure provides a genetically modified strain of Aureobasidium pullulans NRRL 50384 Δpks::ura3 optimized to produce increased quantities of heavy oils by restructuring host metabolism and gene expression to enhance current heavy oil biosynthesis and secretion. The present disclosure presents genetic changes that divert metabolic resources from irrelevant byproducts and also optimize pathways and resources towards extracellular expression of heavy oils.

Accordingly, methods in the present disclosure may include a method of producing a bioproduct comprising growing a genetically modified strain of Aureobasidium pullulans strain under conditions required to support the production of the bioproduct, wherein the genetically modified Aureobasidium pullulans strain comprises an overexpression of a functional VHb protein, and an overexpression of a functional PacC protein; and collecting the bioproduct from the genetically modified strain of Aureobasidium pullulans.

Also described herein is a genetically modified strain of Aureobasidium pullulans, wherein the strain comprises an overabundance of a functional VHb protein; and an overabundance of a functional PacC protein, with optionally a loss of a functional creA gene.

Aureobasidium pullulans is a yeast-like fungus capable of utilizing a plurality of carbon sources and metabolites to produce bioproducts with industrial and pharmaceutical relevance. In some embodiments, Aureobasidium pullulans may utilize a carbon source or renewable feedstock selected from the group consisting of, but not limited to, glucose, xylose, arabinose, sucrose, corn steep liquor, high fructose corn syrup, lignocellulosic hydrolysates, agricultural sugars such as corn sugar, conventional sugars, waste sugar, syrup from corn grind ethanol facilities, or a combination thereof.

Within the diverse set of bioproducts of Aureobasidium pullulans are the surface-active molecules biosurfactants, a secondary metabolite that is well studied in therapeutics and the bioremediation of organic and inorganic pollutants. Within biosurfactants are glycolipid molecules with amphiphilic moieties that are heavier than water, classified as heavy oils.

Extracellular heavy oils may broadly include amphiphilic polyol lipids and aglycone oligo-dihydroxydecanoic acids (DDA) (previously exophilins) and may be comprised of 3,5-dihydroxydecanoyl and/or 5-hydroxy-2-decenoyl esters of a polyol head group such as arabitol, glycerol, or mannitol. In one or more embodiments, the heavy oil may comprise a mannitol head group attached to polyester tail of a repeat of at least three and up to five 3,5-dihydroxydecanoyl ester groups by ester bond (Formula 1). The 3,5-dihydroxydecanoyl ester groups within heavy oil may exist in non-acetylated, monoacetylated, or peracetylated forms where hydroxyl groups can undergo O-acetylation.

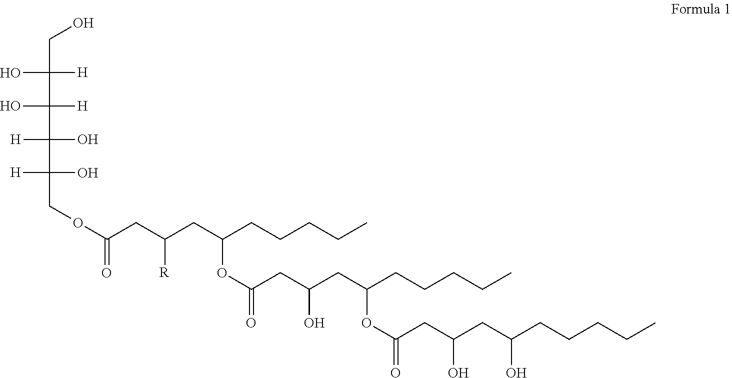

Current paradigms of heavy oil production by Aureobasidium pullulans strains suffer from titers limited to not higher than 22 g/L at maximum titer. This may be due in part to heavy oils competing with other cellular side byproducts such as pullulan and poly-malic acid (PMA) for molecular resources such as glucose and oxygen used for energy (ATP) production. Methods for reducing the consumption of metabolic resources by unrelated bioproducts may include reprogramming the metabolic network for a strain of Aureobasidium pullulans through genetic editing.

The genetic editing of target genes within a strain of Aureobasidium pullulans DNA may be carried out by any means necessary, and by no particular method. In some embodiments, the editing of the genomic DNA of a strain of Aureobasidium pullulans may include, but is not limited to, the interruption, deletion, down-regulation, or a knockdown of a portion of or all of a target gene, the multiplication of a target gene, the induced expression, overexpression, or constitutive expression of a gene, or any combination thereof. The editing of target genes may be completed by any methods known to those skilled in the art and may include, but is not limited to, methods that use homologous recombination, methods that use phage recombinases, methods that use phage integrases, methods that use CRISPR/Cas technologies, or methods that use transposases. In various embodiments, a deletional mutation may be carried out by a linear DNA cassette. Linear DNA cassettes can be grouped into two general classes where they facilitate gene knockouts and gene overexpression (gene knockout cassette and gene overexpression cassettes, respectively). For example, with linear DNA knockout casettes, wherein the knockout cassette comprises isolated nucleic acid sequences that are homologous to the 5′ and 3′ regions of the target gene, a selective antibiotic marker gene, and a promoter DNA sequence, all flanked by two restriction enzyme attachment sites. In a similar fashion to the linear DNA cassette, multiplying the copy number of a target gene may include a linear DNA overexpression cassette. In various embodiments, the linear DNA (e.g., gene) overexpression cassette comprises an addition of a promoter and target gene into the deletional knockout cassette, or additionally or alternatively ribosome binding sites or any other gene regulation sequences. In one or more embodiments, an overexpression mutation may be carried out by the manipulation of the promoter and/or ribosome binding site (RBS) sequence of the target DNA or an overexpression cassette, including, but not limited to, promoter swapping and/or RBS swapping.

In a non-limiting example, the knockout cassette may target the creA gene to render creA non-functional. In another non-limiting example, the knockout cassette may target the PMA synthase enzyme gene to disable the function PMA synthase and/or target the UDPG-pyrophosphorylase enzyme to disable its function.

Methods of the genetic alteration to the strain of Aureobasidium pullulans through cloning may be assembled and carried out in any manner or fashion that is known to one skilled in the art. In one or more embodiments, the genetic alteration is completed through homologous recombination. In various embodiments, the genomic editing methods may be selected from the group consisting of restriction enzyme ligation, Gateway cloning, Gibson Assembly homologous recombination, Golden Gate Assembly, TOPO (TA) cloning, and the like, or any combination thereof.

In a non-limiting example, the genetic alteration of the strain of Aureobasidium pullulans is conducted by homologous recombination, wherein a linear DNA cassette targeting a gene within the strain of Aureobasidium pullulans is cloned using Gibson Assembly to introduce an antibiotic resistance marker flanked by ΦC31 attachment sites, a promoter, an RBS, a terminator, a target gene, or a combination thereof. Following recombination, the serine phage recombinase ΦC31 enzyme recombines the linear DNA cassette at the attachment sites, removing the antibiotic resistance marker and leaving behind a single attachment scar either with or without the target gene.

The insertion and integration of genetic editing cassettes into the strain of Aureobasidium pullulans may be carried out by any genetic transformation techniques familiar to one skilled in the art. In some embodiments, techniques may be selected from, but not limited to, heat shock, chemical transformation, electroporation, conjugation, spheroplast transformation and LiAc/ssDNA/PEG transformation, and any combination thereof. Transformation of the strain of Aureobasidium pullulans would produce a genetically modified strain of Aureobasidium pullulans.

To enhance heavy oil yield, a plurality of genes for proteins and enzymes coding for cellular byproducts outside the biosynthesis of heavy oils, such as PMA and pullulan, may be deleted, knocked out, or down-regulated.

In a non-limiting example, the genetically modified strain of Aureobasidium pullulans is transformed with a knockout cassette targeting the PMA synthase enzyme gene and/or the UDPG-pyrophosphorylase enzyme gene. The management of endogenous metabolites may dictate the amount of bioproduct that can be produced. In fungi, carbon resources such as glucose are regulated by the transcription factor creA through carbon catabolite repression (carbohydrate metabolism) by the control of enzyme-encoding genes required for prioritizing alternative carbon sources. The loss of the creA transcription factor may relieve the repression of glucose as a carbon resource.

In a non-limiting example, the genetically modified strain of Aureobasidium pullulans is transformed with a knockout cassette targeting the creA gene. Restructuring of microbial energetic resources, such as ATP, NAD+/NADH, and NADP+/NADPH, may also improve flux toward targeted bioproducts. Utilization of available metabolic resources used for energetic maintenance, such as oxygen, requires proteins that couples to and deliver the metabolite to relevant cellular process machinery. The introduction and overexpression of the oxygen-binding protein Vitreoscilla hemoglobin (VHb) gene may transcribe an excess of protein that enhances acetyl-CoA-independent production of ATP, thereby increasing the production of bioproducts such as enzymes, amino acids, biofuels, and other polymers. In some embodiments, a functional VHb protein may include any VHb protein that has at least 90% amino acid sequence homology with VHb (the sequence set forth in SEQ ID NO. 1), and functions as a soluble heme-binding protein. A preferred VHb protein is that of SEQ ID NO. 1, but it is understood by those of skill in the art that this sequence could vary by as much as 10% in sequence homology and still retain the equivalent characteristics that render it useful in the present methods.

Though the biosynthetic pathway of heavy oils has historically been characterized by the key gene cluster GAL1-EST1-PKS1, the pH signaling transcription factor PacC has previously been noted as a major expression regulator of the key gene cluster, particularly at low pH. The pH-responsive PacC transcription factor has been thoroughly studied in numerous fungal species and has been identified in various biosynthetic pathways.

In a non-limiting example, the genetically modified strain of Aureobasidium pullulans is transformed with an overexpression cassette of the vhb gene and/or an overexpression cassette of the pacC gene.

The genetically modified strain of Aureobasidium pullulans may be grown in any number of conditions and mediums known to one skilled in the art. In various embodiments, the genetically modified strain of Aureobasidium pullulans may be grown for a duration of about 2 days to about 10 days, agitated at a speed of about 50 RPM to about 400 RPM, and kept at a temperature range of about 20° C. to 35° C. In a non-limiting example, the genetically modified Aureobasidium strain is grown for seven days in an incubator set at a temperature of 30° C. and agitated at 180 RPM.

In one embodiment, the genetically modified strain of Aureobasidium pullulans is grown in a liquid nutrient solution comprising glucose, polypeptone, yeast extract, or any combination thereof.

The genetically modified strain of Aureobasidium pullulans may be grown in an acidic growth medium. In some embodiments, the pH of the acidic growth medium falls within the range of about pH of 1 to about a pH of 6. In a non-limiting example, the pH of the acidic growth medium is a pH of 3.

For optimal heavy oil yield, the genetically modified strain of Aureobasidium pullulans may be grown in a lipid production medium to promote the production of heavy oil. In some embodiments, the lipid production medium comprises glucose, biomass hydrolysate, corn steep liquor, fructose, high fructose corn syrup, waste sugars, sucrose, xylose, arabinose, syrup from corn grind ethanol facilities, the like, or a combination thereof. In a non-limiting example, the lipid production medium comprises 140.0 g/L of glucose, 0.2 g/L of corn steep liquor, 0.6 g/L of NH₄NO₃, 0.1 g/L of KH₂PO₄, 0.5 g/L of KCl, and 0.2 g/L of MgSO₄·7H₂O.

The genetically modified strain of Aureobasidium pullulans may be grown in a variety of methods and conditions known to one skilled in the art. In some embodiments, the genetically modified strain of Aureobasidium pullulans is grown by fermentation methods such as batch fermentation, fed-batch fermentation, continuous fermentation, repeated fed-batch fermentation, and the like. In a non-limiting example, the Aureobasidium strain is grown by fed-batch fermentation, wherein the Aureobasidium pullulans strain is fed a substrate and supplements, such as mineral salts and nutrients, as the culture reaches the logarithmic phase during the fermentation process. This feeding is controlled and extends the logarithmic phase and attains a higher product yield. In a non-limiting example, the genetically modified strain of Aureobasidium pullulans is grown by 10-liter fermentation. In a non-limiting example, fermentation occurs in a baffled bioreactor over the course of about 7 days to about 12 days. Dissolved oxygen is maintained above 30% with agitation in the range of 250-500 RPM with the carbon substrate feed starting after 24 hours to maintain a steady concentration within the reactor.

The genetically modified strain of Aureobasidium pullulans may be fermented in a bioreactor or fermenter, where the yeast-like fungus is cultured in a controlled system at a plurality of temperatures and pressures. In various embodiments, the genetically modified strain of Aureobasidium pullulans may be grown in a bioprocessing system, a stirred tank bioreactor, a jet loop reactor, an air lift reactor, any combination thereof, or the like. In a non-limiting example, the genetically modified strain of Aureobasidium pullulans is fermented in an Eppendorf BioFlo® 320 reactor.

As the genetically modified strain of Aureobasidium pullulans is fermenting, heavy oils may be secreted and settle at the bottom of the fermentation vessel. The heavy oil is out of phase with the fermentation broth, and may be extracted and collected by any methods and procedures known to one skilled in the art. In some embodiments, the heavy oils may be collected by mechanical cell disruption, gravimetric methods, centrifugation, enzymatic processes, solvent extraction, supercritical fluid solvent extraction, the like, or a combination thereof.

In some embodiments, the heavy oils may be collected from the genetically modified strain of Aureobasidium pullulans by solvent extraction methods utilizing an organic chemical solvent that may include, but is not limited to, methyl ethyl ketone (MEK), ethyl acetate, chloroform, methanol, butanol, isopropanol, the like, and a combination thereof. In some embodiments, fermentation broth is centrifuged and the supernatant is removed, leaving the cell and oil fraction. Solvent is added in a 1:1 ratio of solvent to fermentation broth and vigorously mixed. The oil-containing solvent fraction is collected and the solvent is evaporated, leaving the pure heavy oil product.

In further embodiments, the solvent extraction may utilize a non-polar solvent selected from the group consisting of hexane, toluene, petroleum ether, MEK, acetonitrile, ethyl acetate, and a combination thereof. In some embodiments, the polarity index (PI) of the chemicals in the solvent may range from about PI=0 to about PI=6. In a non-limiting example, approximately 50 ml of yeast-like cells and heavy oil are removed by centrifugation at 10,000×g for 20 min. Extracellular heavy oil appears as a layer beneath the precipitated yeast-like cells. Yeast-like cells are gently resuspended in 3-5 ml of distilled water and transferred to a screw-cap glass tube (13 mm×100 mm). Culture flasks and centrifuge bottles were washed with 3-5 ml of MEK and heavy oil was dissolved in this solvent. The dissolved oil recombines with resuspended cell debris and vortexed thoroughly, the resuspension is left overnight to separate at room temperature (20° C.-22° C.). The extracted yeast-like cell debris layer is removed, and the solvent is removed from the heavy oil by steamed air evaporation. Cells are lysed through exposure to MEK, which serves to liberate any endohenous heavy oil. In another non-limiting example, the heavy oil is extracted by whole culture method through isolation from the solvent MEK via a separatory funnel.

In one or more embodiment, heavy oils secreted by the genetically modified strain of Aureobasidium pullulans may be collected by supercritical fluid extraction utilizing supercritical fluid solvent including, but not limited to, supercritical CO₂, supercritical H₂O, supercritical methanol, supercritical ethanol, supercritical ethane, supercritical xenon, and the like, and any combination of the aforementioned supercritical fluids combined with a co-solvent including, but not limited to, ethanol, methanol, water, aqueous ethanol-water at about 50% v/v, and aqueous methanol-water at about 70% v/v. In a non-limiting example, the supercritical fluid solvent may have a purity of 90% purity to about 99.9% purity.

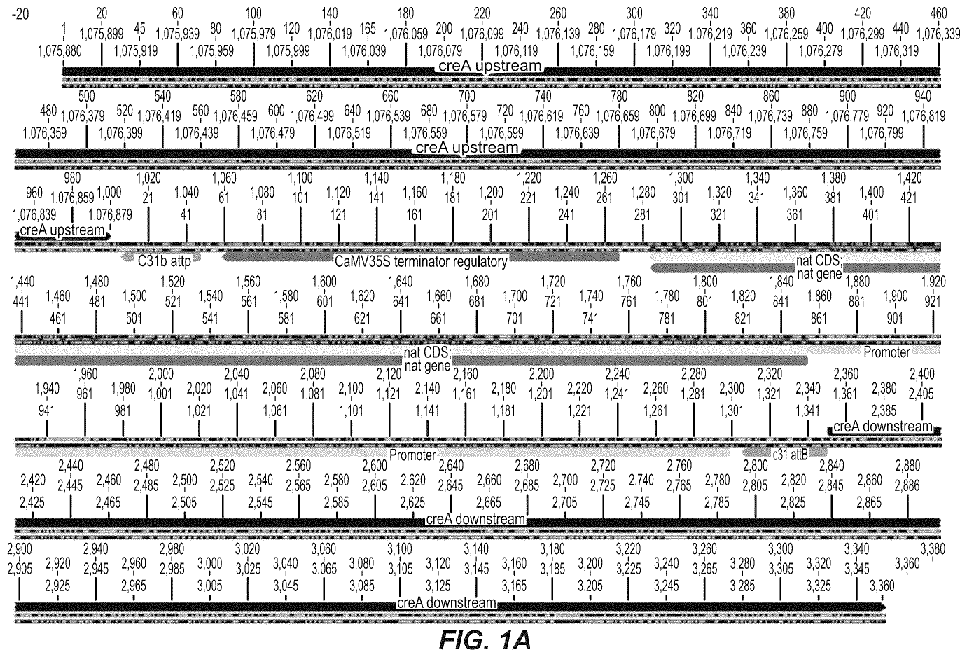

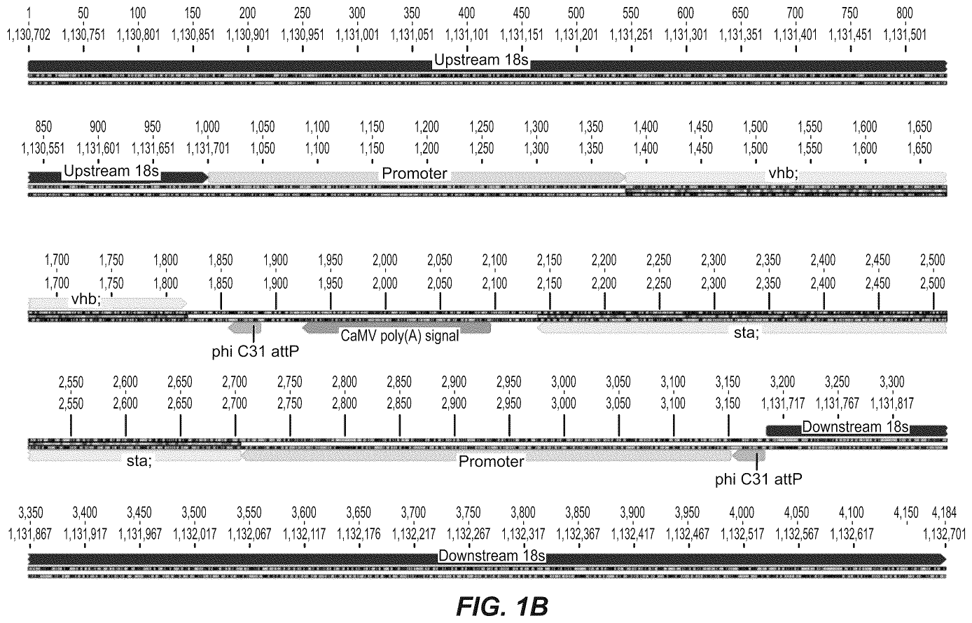

FIG. 1A provides a representative diagram of DNA constructs used for the deletion of the creA gene in Aureobasidium pullulans. The DNA construct is a linear DNA cassette composed of the selective antibiotic marker gene nourseothricin N-acetyl transferase (nat) fused to the CaMV355 terminator regulatory sequence and a promoter sequence, all flanked by a ΦC31 attp and a ΦC31 attB attachment site. At one kilobase upstream and downstream of the antibiotic marker gene, there is encoded sequences with homology to the 5′ and 3′ ends of target creA DNA sequence, respectively. Consequently, transformation of the creA gene deletion cassette into the host strain allows for insertion of the nat gene within the coding sequence of the creA gene via homologous recombination, disrupting functional CreA protein expression.

FIG. 1B provides a representative diagram of DNA constructs used for the overexpression of the vhb gene in Aureobasidium pullulans. The DNA construct is a linear DNA cassette composed of a promoter translation elongation factor (pTEF) fused to the vhb gene sequence inserted downstream of the 18s ribosomal sequence, all flanked by a ΦC31 attp and a ΦC31 attB attachment site

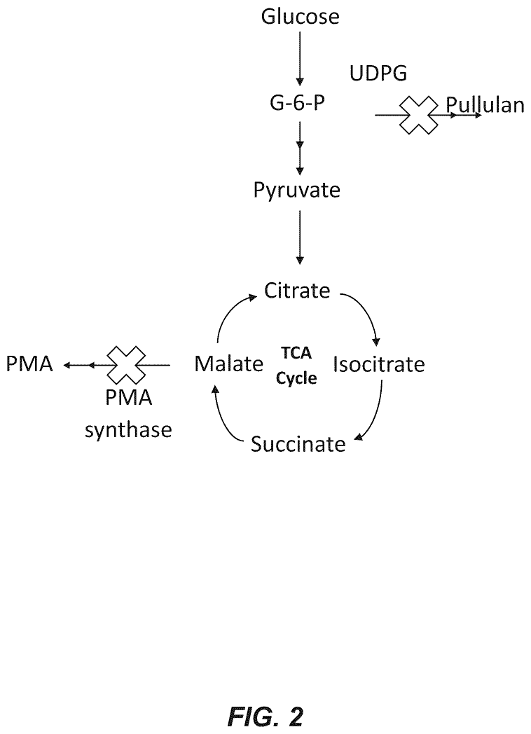

FIG. 2 provides a proposed suppression of biosynthetic pathways of the cellular byproducts poly-malic acid (PMA) and pullulan for the enhancement of heavy oils production in a genetically modified strain of Aureobasidium pullulans. Through the inactivation of these competing byproducts, more metabolic resources are made available for increased yield of heavy oil.

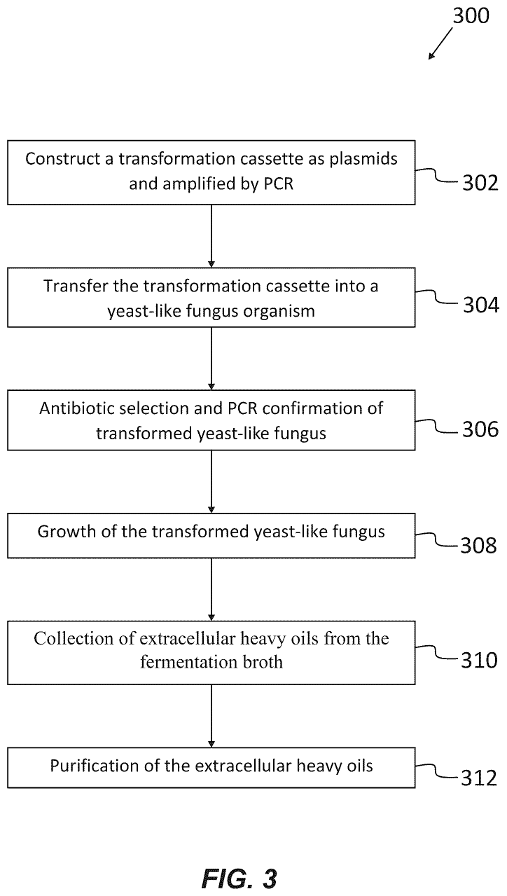

FIG. 3 is a block diagram of a non-limiting system and method of the present disclosure genetically editing a strain of Aureobasidium pullulans and collecting enhanced levels of heavy oils. At block 302, an artificial DNA construct is manufactured and assembled, then amplified by PCR. The construct is a transformation cassette with an antibiotic selection marker. At block 304, the transformation cassette in the form of a linear DNA fragment is transformed into the strain of Aureobasidium pullulans, whereby homologous recombination facilitates the permanent insertion of the artificial DNA into the Aureobasidium pullulans genome.

At block 306, the transformed strain of Aureobasidium pullulans is grown on agar plates containing the selective antibiotic compatible with the antibiotic selection marker of the transformation cassette of block 302. The transformed modified strain of Aureobasidium pullulans that is successfully transformed will flourish while unsuccessfully transformed strains of Aureobasidium pullulans will die off. Furthermore, the presence of the artificial DNA in the strain of Aureobasidium pullulans is confirmed by PCR. At block 308 the antibiotic-selected transformed strain of Aureobasidium pullulans is grown by 10-liter fermentation within a fermenter or bioreactor such as an Eppendorf BioFlo® 320.

At block 310, as the transformed strain of Aureobasidium pullulans is fermented, it produces extracellular heavy oils, such as liamocins, and these heavy oils are collected from the fermentation broth by centrifugation and/or extraction by no particular method. At block 312 the collected heavy oils are purified by any means or methods known to one skilled in the art.

While various embodiments have been shown and described herein, modifications may be made by one skilled in the art without departing from the scope of the present disclosure. The embodiments described here are exemplary only and are not intended to be limiting. Many variations, combinations, and modifications of the embodiments disclosed herein are possible and are within the scope of the disclosure. Accordingly, the scope of protection is not limited by the description set out above, but is defined by the claims which follow, that scope including all equivalents of the subject matter of the claims.

### EXAMPLES

### Example 1: DNA Cassette Assembly

The parental strain used in the examples was Aureobasidium pullulans NRRL 50384 (Δpks::ura3) described in U.S. Pat. Nos. 10,351,889 and 10,612,063). Aureobasidium pullulans natively produces a black pigment, melanin, and the gene responsible for this, polyketide synthase (pks), has been replaced with orotidine 5-phosphate decarboxylase in the strain NRRL 50384 (Δpks::ura3). The disclosure of U.S. Pat. Nos. 10,351,889 and 10,612,063 with regard to the strain is incorporated herein by reference in its entirety.

Gene Knockout Cassettes. Gene Knockout cassettes were assembled as plasmids by Gibson assembly. Each DNA cassette is composed of a nourseothricin N-acetyl transferase (nat) gene for antibiotic resistance, flanked by ΦC31 attachment sites. Outside of the antibiotic marker lies a 1 kilobase of homology upstream and downstream of a target DNA region. The plasmid backbone contains an E. coli origin of replication, pUC, and the kanamycin antibiotic resistance gene. Knockout cassettes were assembled for the following target genes: three variation of UDP-phosphorylate gene (SEQ ID NO. 4-6).

Gene Overexpression Cassettes. Gene Overexpression cassettes were constructed using Gibson assembly by modification of a gene knockout cassette. A target DNA sequence was fused to the pTEF promoter (SEQ ID NO. 8) and inserted downstream of the 18s ribosomal sequence, with 1000 base pair homology to the downstream region of 18s (FIG. 1B). Overexpression cassettes were assembled for the target genes pacC (SEQ ID NO. 9) and the Vhb (SEQ ID NO. 2).

### Example 2: Construction of Aureobasidium pullulans Strain

Strain Construction and Transformation. The DNA cassettes assembled in Example 1 were amplified by PCR to produce linear DNA and were prepared for transformation into an electrocompetent strain of Aureobasidium pullulans NRRL 50384 (Δpks::ura3) for the construction of a genetically modified strain of Aureobasidium pullulans. DNA cassettes were constructed using Gibson assembly with nourseothricin N-acetyl transferase (“nat”) antibiotic resistance markers flanked by ΦC31 attachment sites and 1 kilobase homology regions.

Competent Cell Preparation. The parental strain of Aureobasidium pullulans NRRL 50384 (Δpks::ura3) was grown in 50 mL of Aureobasidium growth medium to an optical density at 600 nm (OD₆₀₀) of about 2 to about 4. The growth medium contained 20 g/L of glucose, 20 g/L of polypeptone, and 10 g/L of yeast extract. The parental strain was then centrifuged for 5 minutes at 4° C. and 5,000 RPM. Once pelleted, the supernatant was removed, and the Aureobasidium pullulans cells were washed twice with Buffer 1 (composed of 1 M sorbitol and 50 mM sodium citrate), then resuspended in 1 mL of cold 10% glycerol.

Electroporation. The competent Aureobasidium pullulans NRRL 50384 (Δpks::ura3) were transformed by mixing 60 μl of Aureobasidium pullulans cells with 6 μl of linear DNA within a 2 mm cuvette. The cuvette was then electroshocked at 200Ω resistance, 25 F capacitance, and 1.5 kV voltage. The electroshocked Aureobasidium pullulans was then recovered from the cuvette by resuspension with 1 mL of Aureobasidium growth media for 4 hours.

Selection and Confirmation. For the confirmation of successful transformation of linear DNA, transformed cells were selected on agar plates containing 50 g/ml nourseothricin and verified by PCR amplification of inserted linear DNA.

Antibiotic Marker Removal. The successful transformation of DNA plasmids containing ΦC31 attachment sites in Aureobasidium pullulans NRRL 50384 (Δpks::ura3) were then selected for the removal of the antibiotic resistance marker gene nat. Transformed Aureobasidium pullulans were grown on agar plates containing 200 g/ml hygromycin antibiotic. Colonies were then selected and streaked on plates containing no selection antibiotic and nourseothricin antibiotic to determine the successful extraction of the antibiotic resistance gene. The revised protocols document eliminates the redundant paragraph that repeated the same experimental conditions and consolidates the information into a clearer, more organized format without duplication.

### Example 3: Fermentation of the Genetically Modified Strain of Aureobasidium Pullulansand Production of Heavy Oil

The strains in these Examples were tested in shake flask experiments, conducted in duplicate or triplicate with appropriate parental controls under identical conditions, as discussed below.

Fermentation conditions. For the production and extraction of heavy oil, the genetically modified strains of Aureobasidium pullulans according to the present disclosure, were grown by shake flasks at 28° C. for 10 days in 50 mL of oil production medium.

Heavy oil extraction and purification. Heavy oil secreted by the genetically modified strains of Aureobasidium pullulans according to the present disclosure were extracted and purified by MEK solvent extraction. After growth, the fermentation broth was centrifuged for 5 minutes at a speed of 5,000 RPM. The supernatant was removed, then the Aureobasidium pullulans cells were resuspended by a 1:1 MEK solvent solution via vortex, causing lysis of the cells. The Aureobasidium pullulans cell debris and MEK mixture were then centrifuged for 5 minutes at 5,000 RPM, and the MEK with the dissolved heavy oil was transferred to a 100 mL round bottom flask. The MEK was then separated from the heavy oil by rotary evaporation. The remaining heavy oil was then quantified by weight.

Analytical methods. The quantification of side bioproducts such as glucose, pullulan, and PMA secreted by the Aureobasidium pullulans within the fermentation broth was carried out by high-performance liquid chromatography (HPLC). A Waters™ Arc HPLC system was equipped with an Aminex-HPX-87H column (Bio-Rad). The HPLC mobile phase was 5 mM sulfuric acid under isocratic conditions at 60° C.

### Example 4. Vhb Overexpression

In Example 4, the heterologous gene encoding Vitreoscilla hemoglobin, vhb, is constitutively expressed to increase heavy oil titers. The production of heavy oil is an oxygen intensive process and overexpression of Vhb increases oxygen availability for the pathway through binding of oxygen.

The VhB enzyme (SEQ. ID No. 1) was overexpressed in Aureobasidium pullulans NRRL 50834 Δpks::ura3strain through insertion downstream of the 18S ribosomal sequence. To overexpress the enzyme, a linear DNA cassette was constructed with 1000 bp of homology to the downstream region of the 18S DNA sequence (Image 1). A promoter (pTEF) and vbh were inserted into the cassette upstream of the antibiotic resistance marker.

The strain was tested in shake flask experiments in duplicate. The VhB overexpression strain and the parental control strain(Δpks::ura3) where each grown in duplicate in 50 mL shake flasks containing a minimal oil production media. Glucose was quantified using HPLC.

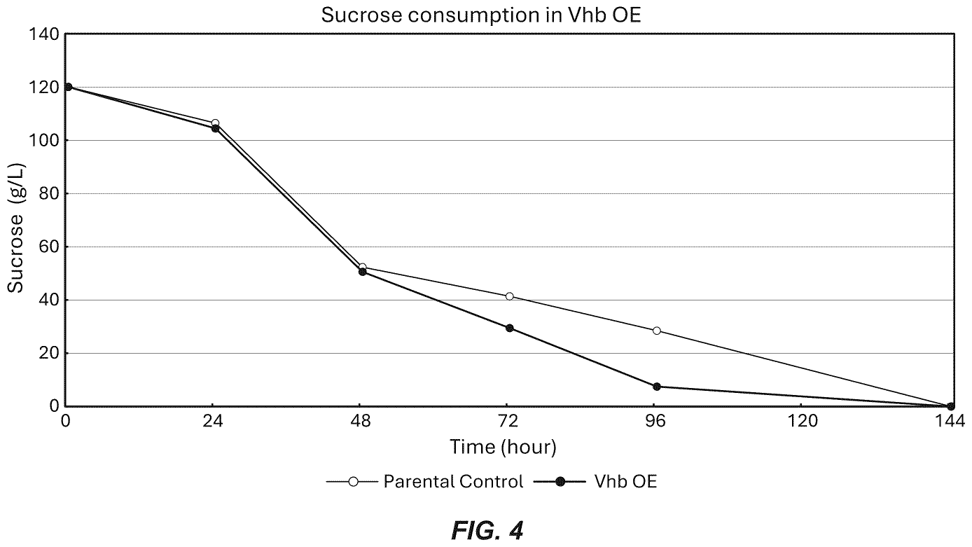

FIG. 4 depicts sucrose consumption in VhB overexpression strains, depicting both the parental control and an average of the VhB overexpression strains produced. The VhB overexpression strain demonstrated a 22% increase in sucrose consumption in Aureobasidium. pullulans, e.g., comparison to the parental control.

### Example 5. PACC Overexpression

In Example 5, the native pacC gene is overexpressed to increase heavy oil titers. PacC regulates expression of gene members of the heavy oil production pathway. Overexpression of pacC increases expression of the heavy oil synthesis pathway, increasing heavy oil titers.

The strain was tested in shake flasks in duplicate. The pacC overexpression strain was grown in duplicate with the parental control (Δpks::ura3) in 50 mL shake flasks containing a minimal oil production media.

Glucose and pullulan were quantified using HPLC. The heavy oil was quantified through solvent extractions. The entire culture was spun down at 5,000×g for 5 minutes. The supernatant was decanted and the cell and oil layer was mixed with 25 mL MEK. The MEK mixture was then spun down and the MEK/oil solution was transferred to a round bottom flask. The MEK was evaporated off with a rotary evaporator and the remaining oil was weighed.

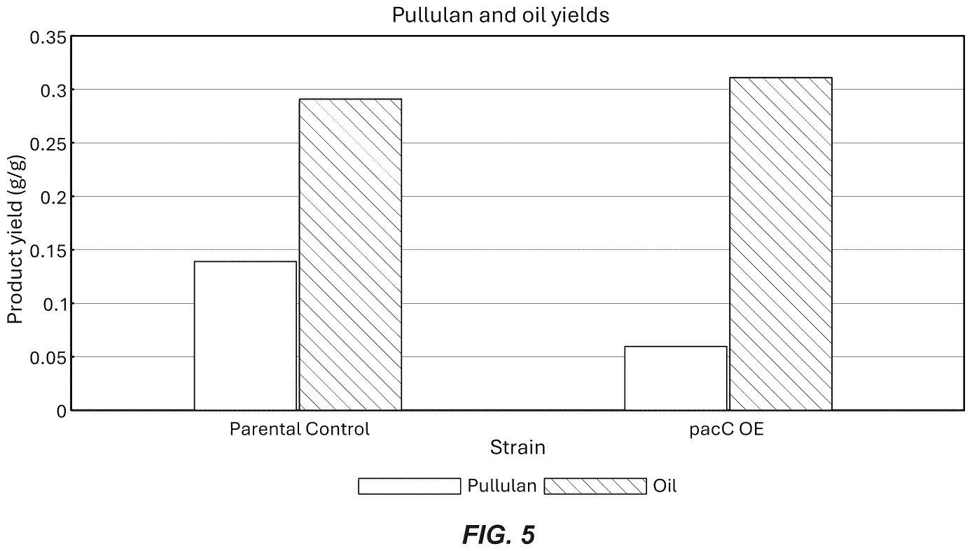

Shown in FIG. 5, The pacC overexpression strain demonstrated a 60% decrease in pullulan yield on a gram pullulan/gram glucose consumed basis in comparison to the parental strain. Oil yields are comparable between the two strains with the pacC strain with a 7% higher yield.

### Example 6. Pullulanase Overexpression

In Example 6, the gene responsible for pullulan degradation (pullulanase) into sugar monomers was overexpressed. The overexpression of this gene allowed for conversion of pullulan into heavy oil.

The pullulanase enzyme (SEQ. ID No. 10) was overexpressed in Aureobasidium pullulans NRRL 50834 Δpks::ura3 strain through insertion downstream of the 18S ribosomal sequence. To overexpress the enzyme, a linear DNA cassette was constructed with 1000 bp of homology to the downstream region of 18S. A promoter (pTEF) and pullulanase were inserted into the cassette upstream of the antibiotic resistance marker.

The strain was tested in shake flask experiments in duplicate. The pullulanase overexpression strain and the parental control (Δpks::ura3) strain were each grown in duplicate using 50 mL shake flasks containing a minimal oil production media. Glucose and pullulan were quantified using HPLC.

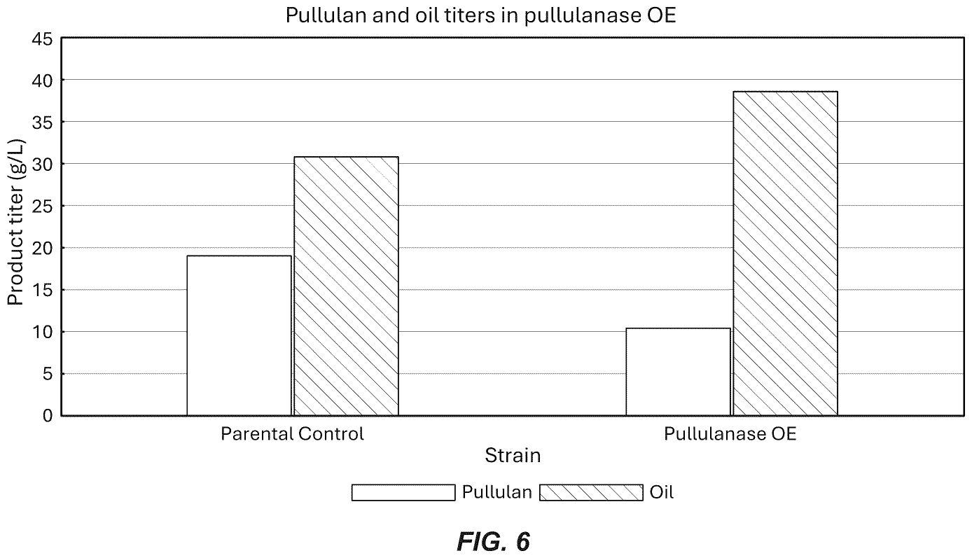

FIG. 6 depicts pullulan and oil titer in pullulanase overexpression strains. Shown are both the parental control and the pullulanase overexpression strain, with results for both pullulan and oil production in g/L. The pullulanase overexpression strain demonstrated 45% decrease in final pullulan titers and 25% increase in heavy oil in comparison to the wild type.

### Example 7. Pullulan Production Knockout

Production of competing byproduct, pullulan, was reduced to direct carbon towards heavy oil production and streamline downstream processing. Accordingly, the genes encoding enzymes that catalyze the first step of pullulan production (UDPG-pyrophosphorylase) were knocked out.

In Example 7, three UDP-phosphorylase gene sequences were identified, UDP-phosphorylase Nucleotide Sequence #1-3 below (SEQ. ID Nos. 4, 5, 6). DNA cassettes were constructed as plasmids through Gibson assembly. The cassette contained a nourseothricin N-acetyl transferase (nat) gene for antibiotic resistance flanked with ΦC31 attachment sites. Outside of the antibiotic marker there was 1 kilobase of homology upstream and downstream of the target DNA region. The plasmid backbone contained an E. coli origin of replication, pUC, and the kanamycin antibiotic resistance gene.

The cassette was PCR amplified to produce linear DNA and transformed into Aureobasidium pullulans. For transformation, electrocompetent Aureobasidium pullulans cells were prepared. The parent strain was grown in 50 mL of Aureobasidium growth media until an OD₆₀₀ of 2 to 4 was reached. The cells were then centrifuged at 4° C. for five minutes at 5,000 RPM. Supernatant was decanted and the cells are washed twice with a buffer of sorbitol and sodium citrate. Cells were resuspended after the spin in 1 mL of cold 10% glycerol. For electroporation, 60 μL of cells are mixed with 6 μL of linear DNA in a electroporation cuvette. The cuvette was shocked using a Bio-Rad electroporator set to 200Ω resistance, 25 μF capacitance, and 1.5 kV voltage. The transformed cells were recovered in 1 mL of the Aureobasidium growth media for four hours. Insertion events were selected on 50 μg/mL nourseothricin agar plates and confirmed through PCR of the region. A plasmid containing ΦC31 is transformed into Aureobasidium pullulans to remove the antibiotic resistance marker. Cells were selected on 200 μg/mL hygromycin agar plates. Colonies were streaked on agar plates containing no selection. Streaked colonies were patched on non-selective plates and nourseothricin plates to identify cells that had lost nat.

Heavy oil was quantified using a MEK solvent extraction procedure. The fermentation broth from the shake flasks was spun down for 5 minutes at 5,000 RPM. Supernatant was removed and the cells were resuspended in 1:1 ratio of MEK by vortex. The MEK/cell mixture was then centrifuged for 5 minutes at 5,000 RPM and the MEK with dissolved oil was transferred to a 100 mL round bottom flask. MEK was removed via rotary evaporation and the remaining oil was weighed. Pullulan production was determined by shake flask growth experiments. The three knockout strains and the parental strain were grown in shake flasks with 50 mL of oil production media at 28° C. for 10 days. The media is comprised of 120.0 g/L of glucose, 0.2 g/L of corn steep liquor, 0.6 g/L of NH₄NO₃, 0.1 g/L of KH₂PO₄, 0.5 g/L of KCl, and 0.2 g/L of MgSO₄·7H₂O.

Oil, PMA, and pullulan production were determined by shake flask growth experiments. Here, the engineered strains and the parental strain were grown in shake flasks with 50 mL of oil production media at 28° C. for 10 days.

Glucose, pullulan, and PMA in the fermentation broth were quantified using high-performance liquid chromatography (HPLC). A Waters Arc system with used equipped with an Aminex-HPX-87H column (Bio-Rad). The mobile phase was 5 mM sulfuric acid under isocratic conditions at 60° C.

Glucose and pullulan in the fermentation broth were quantified using high-performance liquid chromatography (HPLC). A Waters Arc system is equipped with an Aminex-HPX-87H column (Bio-Rad). The mobile phase was 5 mM sulfuric acid under isocratic conditions at 60° C.

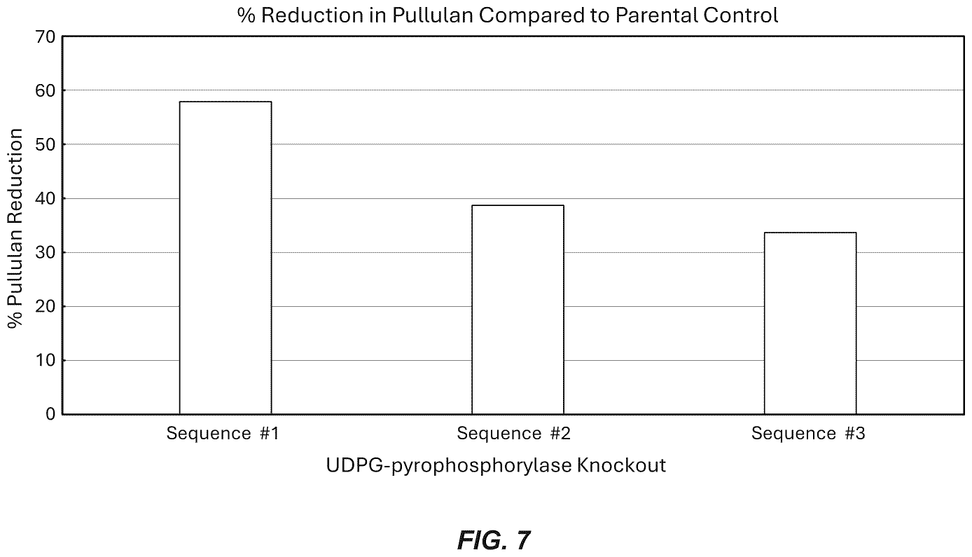

The pullulan production of the three strains was compared to the parental strain Aureobasidium pullulans NRRL 50834 Δpks::ura3 on a g pullulan/g glucose consumed basis. These results are depicted in the chart of FIG. 7. Here, the percent in pullulan reduction is illustrated for each of the knockout sequences 1, 2, and 3. Overall, knockout of sequence #1-3 demonstrated a 58%, 39%, and 34% reduction in pullulan, respectively.

### Example 8. Carbon Dioxide Conservation

In Example 8, a combination of genes encoding Aureobasidium melanogenum 9-1 phosphoketolase (PK) and PTA from below were integrated into the chromosome and an experiment will be run to determine if there is an increase of heavy oil. See SEQ. ID Nos. 11 to 14.

### Non-limiting Example Embodiments

### Additional Embodiments Disclosed Herein Include

Embodiment A: a method of producing a bioproduct comprising: growing a genetically modified strain of Aureobasidium pullulans under conditions required to support the production of the bioproduct, wherein the genetically modified strain of Aureobasidium pullulans comprises an overexpression of a functional VHb protein having at least 90% sequence homology with the sequence set forth in SEQ ID NO. 1, and an overexpression of a functional PacC protein; and collecting the bioproduct from the genetically modified strain of Aureobasidium pullulans.

Embodiment B: a composition comprising a strain of Aureobasidium pullulans, wherein the strain comprises: an overabundance of a functional VHb protein having at least 90% sequence homology with the sequence set forth in SEQ ID NO: 1; and an overabundance of a functional PacC protein, with optionally overexpression of pullulanase.

By way of non-limiting example, exemplary combinations applicable to Embodiments A through B include:

Element 1: wherein the genetically modified strain of Aureobasidium pullulans further comprises an overexpression of pullulanase.

Element 2: wherein the genetically modified strain of Aureobasidium pullulans is derived from Aureobasidium pullulans NRRL 50834 Δpks::ura3.

Element 3: wherein the genetically modified strain of Aureobasidium pullulans is modified by a serine phage recombinase enzyme ΦC31.

Element 4: wherein the genetically modified strain of Aureobasidium pullulans lacks a cellular byproduct comprising poly-malic acid (PMA), pullulan, or a combination thereof.

Element 5: wherein the genetically modified strain of Aureobasidium pullulans lacks function of a UDPG-pyrophosphorylase enzyme.

Element 6: wherein the bioproduct is a biosurfactant comprising glycolipids, liamocins, aglycone oligo-dihydroxydecanoic acids (DDA), or a combination thereof.

Element 7: wherein the bioproduct comprises of 3,5-dihydroxydecanoyl and/or 5-hydroxy-2-decenoyl esters of arabitol and mannitol.

Element 8: wherein the genetically modified strain of Aureobasidium pullulans is grown in a lipid production medium comprising glucose, lignocellulosic hydrolysate, corn steep liquor, fructose, high fructose corn syrup, waste sugars, sucrose, xylose, arabinose, syrup from corn grind ethanol processes, or a combination thereof.

Element 9: wherein the genetically modified strain of Aureobasidium pullulans is grown by agitation at 180 RPM for seven days at a temperature of 30° C.

Element 10: wherein the bioproduct comprises a single head group comprising mannitol, arabitol, xylitol, threitol, sorbitol, galactitol, or glycerol.

Element 11: wherein the bioproduct is collected by mechanical cell disruption, centrifugation, solvent extraction, supercritical fluid extraction, or a combination thereof.

Element 12: wherein the bioproduct is collected by the solvent extraction, and wherein the solvent comprises an organic chemical solvent selected from the group consisting of chloroform, methanol, butanol, isopropanol, and a combination thereof, or a non-polar solvent selected from the group consisting of hexane, toluene, petroleum ether, methyl ethyl ketone (MEK), acetonitrile, ethyl acetate, and a combination thereof.

Element 13: wherein the genetically modified strain of Aureobasidium pullulans is grown by batch fermentation, fed-batch fermentation, continuous fermentation, repeated fed-batch fermentation, or a combination thereof.

Element 14: wherein the genetically modified strain of Aureobasidium pullulans is grown in a bioprocessing system, a stirred tank bioreactor, a jet loop reactor, an air lift reactor, or a combination thereof.

Element 15: wherein the genetically modified strain of Aureobasidium pullulans is grown by fermentation in an acidic growth medium.

Element 16: wherein the strain of Aureobasidium pullulans is a genetically modified strain of Aureobasidium pullulans NRRL 50834 Δpks::ura3.

Element 17: wherein the strain of Aureobasidium pullulans produces a heavy oil with titers exceeding 30 g/L.

Element 18: wherein the strain of Aureobasidium pullulans lacks function of a UDPG-pyrophosphorylase enzyme.

By way of non-limiting example, exemplary combinations applicable to A through B include one, more, or all of Elements 1-18, without limitation.

To facilitate a better understanding of the embodiments described herein, the following examples of various representative embodiments are given. In no way should the following examples be read to limit, or to define, the scope of the present disclosure.

The terminology used herein is for the purpose of describing particular embodiments only and is not intended to be limiting of the invention. As used herein, for example, the singular forms “a,” “an,” and “the” are intended to include the plural forms as well, unless the context clearly indicates otherwise. It will be further understood that the terms “contains”, “containing”, “includes”, “including,” “comprises”, and/or “comprising,” and variations thereof, when used in this specification, specify the presence of stated features, integers, steps, operations, elements, and/or components, but do not preclude the presence or addition of one or more other features, integers, steps, operations, elements, components, and/or groups thereof.

Terms of orientation used herein are merely for purposes of convention and referencing and are not to be construed as limiting. However, it is recognized these terms could be used with reference to an operator or user. Accordingly, no limitations are implied or to be inferred. In addition, the use of ordinal numbers (e.g., first, second, third, etc.) is for distinction and not counting. For example, the use of “third” does not imply there must be a corresponding “first” or “second.” Also, if used herein, the terms “coupled” or “coupled to” or “connected” or “connected to” or “attached” or “attached to” may indicate establishing either a direct or indirect connection and is not limited to either unless expressly referenced as such.

While the disclosure has described several exemplary embodiments, it will be understood by those skilled in the art that various changes can be made, and equivalents can be substituted for elements thereof, without departing from the spirit and scope of the invention. In addition, many modifications will be appreciated by those skilled in the art to adapt a particular instrument, situation, or material to embodiments of the disclosure without departing from the essential scope thereof. Therefore, it is intended that the invention not be limited to the particular embodiments disclosed, or to the best mode contemplated for carrying out this invention, but that the invention will include all embodiments falling within the scope of the appended claims. Moreover, reference in the appended claims to an apparatus or system or a component of an apparatus or system being adapted to, arranged to, capable of, configured to, enabled to, operable to, or operative to perform a particular function encompasses that apparatus, system, or component, whether or not it or that particular function is activated, turned on, or unlocked, as long as that apparatus, system, or component is so adapted, arranged, capable, configured, enabled, operable, or operative.

1. A method of producing a bioproduct comprising:
growing a genetically modified strain of Aureobasidium pullulans under conditions required to support the production of the bioproduct,
wherein the genetically modified strain of Aureobasidium pullulans comprises an overexpression of a functional VHb protein having at least 90% sequence homology with the sequence set forth in SEQ ID NO. 1, and an overexpression of a functional PacC protein; and
collecting the bioproduct from the genetically modified strain of Aureobasidium pullulans.

2. The method of claim 1, wherein the genetically modified strain of Aureobasidium pullulans further comprises an overexpression of pullulanase.

3. The method of claim 1, wherein the genetically modified strain of Aureobasidium pullulans is derived from Aureobasidium pullulans NRRL 50384 Δpks::ura3.

4. The method of claim 1, wherein the genetically modified strain of Aureobasidium pullulans is modified by a serine phage recombinase enzyme ΦC31.

5. The method of claim 1, wherein the genetically modified strain of Aureobasidium pullulans lacks a cellular byproduct comprising poly-malic acid (PMA), pullulan, or a combination thereof.

6. The method of claim 1, wherein the genetically modified strain of Aureobasidium pullulans lacks function of a UDPG-pyrophosphorylase enzyme.

7. The method of claim 1, wherein the bioproduct is a biosurfactant comprising glycolipids, liamocins, aglycone oligo-dihydroxydecanoic acids (DDA), rhamnolipin, or a combination thereof.

8. The method of claim 7, wherein the bioproduct comprises of 3,5-dihydroxydecanoyl and/or 5-hydroxy-2-decenoyl esters of arabitol and mannitol.

9. The method of claim 7, wherein the genetically modified strain of Aureobasidium pullulans is grown in a lipid production medium comprising glucose, lignocellulosic hydrolysate, corn steep liquor, fructose, high fructose corn syrup, waste sugars, sucrose, xylose, arabinose, syrup from corn grind ethanol processes, or a combination thereof.

10. The method of claim 1, wherein the genetically modified strain of Aureobasidium pullulans is grown by agitation at 180 RPM for seven days at a temperature of 30° C.

11. The method of claim 7, wherein the bioproduct comprises a single head group comprising mannitol, arabitol, xylitol, threitol, sorbitol, galactitol, or glycerol.

12. The method of claim 7, wherein the bioproduct is collected by mechanical cell disruption, centrifugation, solvent extraction, supercritical fluid extraction, or a combination thereof.

13. The method of claim 12, wherein the bioproduct is collected by the solvent extraction, and wherein the solvent comprises an organic chemical solvent selected from the group consisting of chloroform, methanol, butanol, isopropanol, and a combination thereof, or a non-polar solvent selected from the group consisting of hexane, toluene, petroleum ether, methyl ethyl ketone (MEK), acetonitrile, ethyl acetate, and a combination thereof, or a supercritical solvent.

14. The method of claim 1, wherein the genetically modified strain of Aureobasidium pullulans is grown by batch fermentation, fed-batch fermentation, continuous fermentation, repeated fed-batch fermentation, or a combination thereof.

15. The method of claim 14, wherein the genetically modified strain of Aureobasidium pullulans is grown in a bioprocessing system, a stirred tank bioreactor, a jet loop reactor, an air lift reactor, or a combination thereof.

16. The method of claim 1, wherein the genetically modified strain of Aureobasidium pullulans is grown by fermentation in an acidic growth medium.

17. A composition comprising a strain of Aureobasidium pullulans, wherein the strain comprises:
an overabundance of a functional VHb protein having at least 90% sequence homology with the sequence set forth in SEQ ID NO: 1; and
an overabundance of a functional PacC protein.

18. The composition of claim 17, wherein the strain of Aureobasidium pullulans is a genetically modified strain of Aureobasidium pullulans NRRL 50384 Δpks::ura3.

19. The composition of claim 17, wherein the strain of Aureobasidium pullulans produces a heavy oil with titers exceeding 30 g/L.

20. The composition of claim 17, wherein the strain of Aureobasidium pullulans lacks function of a UDPG-pyrophosphorylase enzyme.


SEQUENCE LISTING (14 sequences)

SEQ ID NO: 1 - protein, 146 aa, Vitreoscilla stercoraria
MLDQQTINIIKATVPVLKEHGVTITTTFYKNLFAKHPEVRPLFDMGRQESLEQPKALAMTVLAAAQNIENLPAILPAVKKIAVKHCQAGVAAAHYPIVGQELLGAIKEVLGDAATDDILDAWGKAYGVIADVFIQVEADLYAQAVE

SEQ ID NO: 2 - DNA, 2316 bp, Vitreoscilla stercoraria
  source 1..2316: mol_type = genomic DNA
tgtggattaagttttaagaggcaataaagattataataagtgctgctacaccatactgatgtatggcaaaaccataataatgaacttaaggaagaccctcatgttagaccagcaaaccattaacatcatcaaagccactgttcctgtattgaaggagcatggcgttaccattaccacgactttttataaaaacttgtttgccaaacaccctgaagtacgtcctttgtttgatatgggtcgccaagaatctttggagcagcctaaggctttggcgatgacggtattggcggcagcgcaaaacattgaaaatttgccagctattttgcctgcggtcaaaaaaattgcagtcaaacattgtcaagcaggcgtggcagcagcgcattatccgattgtcggtcaagaattgttgggtgcgattaaagaagtattgggcgatgccgcaaccgatgacattttggacgcgtggggcaaggcttatggcgtgattgcagatgtgtttattcaagtggaagcagatttgtacgctcaagcggttgaataaagtttcaggccgctttcaggacataaaaaacgcaccataaggtggtctttttacgtctgatatttacacagcagcagtttggctgttggccaaaacttgggacaaatattgccctgtgtaagagcccgccgttgctgcgacgtcttcaggtgtgccttgggcaatgatgcgtccgccaccatcgccatcgccaccttcaggacccaaatcaatgatgtaatcggcggttttgatgacatctaaattgtgttcgatgatgacgatgctgttgcctttgccttttaagcgtgtgataacttctagcaataaagcgatgtcggcaaaatgcaaacctgtggttggttcatccaaaatgtacaaagtgcggcctgtatcgcgtttggacaattccaatgccaatttcacccgttgcgcttcaccgcctgataatgtggtcgcgctttgacccaaacgcacataactcaaacccacatccatcaaagtttgtaatttgcgtgataaggtgggaacggcttcaaaaaaagcatgcgcttcggcaaccgtcatttccaatacttggtgaatgtttttgcctttgtacaaaatttccaaagtttcacggttgtagcgtttgccgtggcaaacctcacacggtacatacacatcgggcaagaaatgcatctctacttttaaaacgccatcgccttgacacgcttcgcatcggccgcctttgacgttgaacgagaagcgaccgacgttgtaaccgcgttcacgtgaaacaggcacgcctgcaaacaattcacgaatcggtgtgaatacacctgtataggttgcagggttggagcggggcgtgcgtccaattgggctttggtcgacattgatgaccttgtccaattgctccaagccttgaatctcggtataaggcgatggctcgtctttggtggcacggttcaaatcacgctgcgcgattttggccaaagtatcgttgatcaaagtcgatttgccgctgcctgacacgccagtgatgcacgtcatcaaacctaagggcaattccaaagtcacatcttttaagttttgccccgaagcacctttcaataccaacatgcgttctgcatctttgggcgtgcgctgtgccggaatgtaaatcgcttctttgccgcttaaatagcgtcctgtaatcgattgctcgcatgcggcgattttttcaggtgtatcggcaatcagtaccgcgccgcccaattcacctgcgccaggacccatatccaccacataatcggccgcacgaatggcatcttcgtcatgttcgaccacaatcacactgttgcccaaatcacgcaaatgtttgagcgtgccaagcaaacggtcgttgtcacgttggtgcaaaccaatagacggctcatccaagacgtacatcacgccagttaaacccgaaccgatttggctggccaagcgaatgcgctgcgcttcgccgcctgataaggtttcagcagagcgggcgagtgacaaataattcaagccgacattgattaaaaagcctaagcgttcggtgatttccttcaagactttttcggcaatttgctgtttgttgccgcccaaatccaagttttggaaaaaatccaaagttttgctcaaaggccatgcgctgatatggtgaaattttcgccaccgacatacacaaagcgggcttctttcgcaacgtgcgcgcacagtgtacaagcgcgaagcttcaggcaactcagcaaccgtttccacataaatcggctctagcttgccatgtacacgaatagcacgagcca

SEQ ID NO: 3 - DNA, 1386 bp, Aureobasidium pullulans
  source 1..1386: mol_type = genomic DNA
atgcagcgcacttcgtccggtgtggatttctccagcctcttgaatccagacaactcttcctcggccactcccccttctcaccccagcactccctcgacctacgtcgacgccggtgtccagtcccatccttccatggctgctgctgccaccgtgcccgccatggccgccaacatgcccactcaggaacagactactcagactcctccccagcaggatgtgcgccccgacctaccgcgtccttacaagtgccctctgtgcgacaaggctttccatcgcctcgagcaccagactcgtcacatccgcacccacactggcgagaagccccacgcctgccagttcccgggctgcacaaagcgcttcagtcgttctgatgagctgacccgtcactcgcgcattcacaacaaccccaactcgagacgtggcaacaagcaacatgccgccgccgctgctgccgctgccgctgccgccgccgccgccgctcaagccggaatcatggaccaagctcagcaccaggtcatgcagcagatgaaccacatgatgcctcctccacccaagatgaacagccttgctcactcggccccatcgtctcaactctcgtcgcccaacgtgtcccctcctcactcttacgccagcgcatacacaagcaaccctggctcttacttgacgcgcaacgtgagcggcgatcttagccctgctggtctcaatcaccccatggacatcaacctcttggccacagctgcctcgcaagtcgagcgcgagaactcgctcccttcgcatcacatgggtcttgccagcagacatacgtacggcacccacccttacagccaccatccttactcgactggccgtcttccttctctttcggcttatcactacagctccaaccccacttctcagcccatgtcgcgttcgcactctcacgaggatgacgacccttactcgcaccgcatgaccaagaagtcgcgtcctggctctcctcagtcaactgcaccaccatcgccttcattctctcacgagtccacctcgccaactcccgaccacactcctttggctactccttctcactctcctcggctccgtccgcagtacttccataacggtgtgcaactccccacactccaccacctctcccttcagcaccaaccgcctcccgcccttgcccctgttgagccgtccgctgacggtcaacagccgtacattccgacgcaccacagcggtcttcgcatcagtgagatcatggaacgcgaagacggagctcaacgaaaactgcccatcccgatggtgccatcagccgtcaaagtcaacgatttgctcaacaacaccaacggtttcagcagcggccagtctagcaccactggtagtgtagctggcgacaactcgaattaa

SEQ ID NO: 4 - DNA, 1000 bp, Aureobasidium pullulans
  source 1..1000: mol_type = genomic DNA
atggcagtaagagacaacttgcaccgcgaggggcagttcagagtgctgaccaggttttagaggacaacaagaaggtgcttcaaagacgacacttcctgactatgacccatctgtacgcaccagcaaagtattcaccagacgatccacgactgaacacagcgacagtgctacctttgtcccggcaacaagcgcgctgctggcgactccaaccctaaatacgaaagcacctacgtcttcgtcaatgactacagtgctgtcaaggaggagcaagccgagtatgagcagccagccaatgatggcagtaggtgtcagcgccgccataatgaacaacggacactgatgcgaattaggtctcgccaacactctgttacgcgctgagcccgtgactggcaaatgctatgtcatcaccttttctcctacacacaacttgaccctcgcagacctccaaccctcagaaatcctccctgtcatcaacacctggacccagatctacacaactcacctgtctcccaagtccattctcgctgaagtcgcacaacctactaccattgctcctcatggccaggaccttgccgcgccaaatgcccaatatcgctacatgcagatcttcgagaataagggtgctgcaatgggctgttccaaccctcacccccatggtcaggtctggtgcacaacaggtacgccagaagagcccgctctcgagttagaaagcctgcaaaagtaccgcaaggagcatggcggatcacacatgctggttgactatgccaatctcgagtctgagaagaaggaaagagtcgtatttgagaacgaatcattccttgctgtatgtccctggtgggctacctggccctttgaggtcatgattgtcgctcgcacccacaagcgtgccttggtcgacttcaacgacaaggacagacaggatttagccgaggctatcgctgaagtcactagacgctatgataaccttttcgagacccaattcccttacagtaagttgtga

SEQ ID NO: 5 - DNA, 5820 bp, Aureobasidium pullulans
  source 1..5820: mol_type = genomic DNA
atgtcatcagtttccccatttccaaggggcaaggatgctccacactgcgccaggatacgaccgtcactatgtgtgatccccgatgctgtcgtcgtgtcggatgaaatggtagttaaagactaaacgcctgccccttctcacaacacttgtccgagacttttctgcttcgaacacacagttgctgctcctgtctcacccatcccatccccgctcacaatgccagtcggatcagttctcgtcaccgggtaagtgacgctttgcgccggactgccccgccccgccttgcgctgatgaacgcgacagtggcaccggatacatcggtggattcactgccctcgccctgctcgaggctgactacaaggtcgtcattgtcgataacctgtacaactcgtctgaggaagtcctgagccgcatcgaaaccatctgcggcaagcgtcccatcttctacaacgccgacattaccgacgagaaggccctggagaaggtctttaccgaccaccctgacatcgacaatgtaattcacttcgccgccctcaaggctgtcggcgagtctggagagatccccctcgagtactggcgcgtcaacgttggcggctcaatcgccctgcttcaggccatgacccgccacaatgtcaccaacatcgtcttctcgtcgtctgctacagtctacggtgatgccacacgcttcgagggcatgatccccattcccgaacattgccctctgggtcctacaaacccttacggagacaccaagtatgccattgaaaagctgatcggcacgcacgtcgaagctcagcgcaacaatgcacagaagaaaggcgagtctggtgccaaatggaacggcgctttgttgcgctacttcaaccctgccggcgcacaccctagcggaatcatgggagaggatcctcaaggaatacccttcaacctcttgcccctgctggcccaagtcgccgtcggaaagcgtgaaaagttgcttgtattcggtgacggtaagcagaattaaggccatgcgcacatgagcatacaggactgacgagtgcacagactacggctcgagagatggcaccgcaatccgcgactatatccacgtcatcgatcttgcgcaaggtcatctcaaggcactcaactaccttcgcgagcaccagcccggcgtgcgcgcatggaaccttggtaccggcagaggctcgaccgtcttcgagatggtcaaggctttctcgcacgctgtcggacgcgatctcccatatgaggtcgttgatcgtagacctggcgacgttctgaaccttacagccaacccagctagagccaacgaggaacttggatgggagactaagctctcgctcgaggatgcgtgcagcgacctttggagatggaccgagaacaaccctcaaggctacagacagcaggcgccgaaagagtttgtggacaaggcgattgctgctcgcaagtaggtgggacggcttcaaggacgacggtaatgcttgcagacgattctttctgttttttttcttttgcacgatagatttgttacggacgctttctcaaaaggttacttcaacctagtgaaggccagcagggatcctggctttcgcacacatattatacccagaagtaatcgcaaaagtatgtagaactaatttaatccgctttcggaagtctaaggggttccacgcgaaacgctcacggatatgacgtcaattggcgcaatgacgcgccgttgttaggtcgatggactgaatgtgtctgtcgaccatcaacccagacttggattgttcacaatcacttgtacatctttgcgctatacattcaggaacagcaatatcgcatttgtaatcgcatcacaccggaccatcacaaccaccatggcacccaagagacgcatctctaccccttcggcagcgcagcgcaatcaacaagcgaccatctcgtttagcaagcagggtctcaatcgcgtcaccaagaacactgcccagcgtgactccaaggccaccaagcttgaatcagtatcaattcatgctatcagtcagcccaccaccgccgatgcagcgatcgaagaacaagccgaggccgagcttgtcgacatcaatgcaactaaatctgagcttcagtccggcgacgagcccgacgactacgagaagcgcgccatcaacatcaccaatactcaaatcaaaaaatactggcagcaaaaagaagccgaaaggaaatcaccccgggttcatcagcaaggactcgatgtgtcagaaaagattctgagggagttcgacatgagcggacagtacggcgtaagtcgcacgcaattttctactagacaaacatttgactgatcgtctttcagccctgcattggtattgccagagtcaagcgttggagaagagctaaaagtttgggtctcaatcctcccatcgaagtgctggcagtcttgctcaaggcgagcgacaaggacaagagcagaatgcagcgtgctcatgtcgacgagttgatgagctcgagattcatcgagacataggagaacgataggaccgaacgattatgacagggaggaatacggcacggaccgggattatgcatggctgggagttatatggcgtggagggggcttgcttcaaacgataccaggactgtgggctcatccaagctgggagattcaagacgtatgatgactaggacattgcttgatacaaccgtaatacaaatgacatggaaacaaaacgtgacagggtcgtgacaagatgtgagagtagagtcattggggttgttgtggttggaaaagccgaattcagtggagacgtcgggggctggaagctctggctgccctgacggaccaactttgtccatcgacacgcagcgacacgacatggccgcacccaacaacaatgcagaggcactgattcctcaattcaagttcgaaaggcttctcaaccaaggtatcaacactcgagcgcaccagaaacatgagttgtaacgcgacgacgacgcatcctagaccaagccggacgccgcatcgtcttgcagggcacaatcgcaagtcaacctgcactgctcctcgccgagcgcgccgcctttgattcggaagatgcacacttgtcgaccttcagtagctcgctgtcacacatccagaacctcggcgacaatgacatctaccgctggtacatggcccacagtggtgctgcccaaggaaacccgcccgacttgaagatcaacctcatctacccatgcaccgatcagcacctcaagaagtacgccgcacaagccctccgctacgtgaccgagactcctgacatctatcgcgaccacgttcgtccgtacatgcagactaagcgcgaccaaggacgactgaactgggtattcaacatcctcgacggacgcaccgagcaagaagacgtcctgttccgctcgccgcatgacacaactcccgacgatgagagatacttgttgcttcctgatttaaactgggaccgcaagacgctgggaagcctgcacttgcttgcactcgtcgagcgcagagacatctggagtctccgcgacctcagaaagaagcacgtcacttggctcaagtccatcgttgcagatatcgcaaccactgtttcccatctatacagcatagactcggacatgctcaaatgctatgtgcactgtatgtcgtattcgtcttttttttttcctttcgcgtgtcttctgtttgttctgacaagaccacagatcaaccaacctactaccatttccacatccacattgtccatgtctcgctcgaggctggtgccacacaggccgtgggtaaagctctgtcgcttgacaacatcatttcccaactcgctaccatggctggtgacgaggaagccggcatggacagtgttgatctttctttcactttgggtgaagccagcgagctatggacagacgtgttcctgcctctgaaggagaagcgccttcaatagagatagagtttgatacatcgaacgttcgatcaatgatttaagttatgcttgttttaggcttcttttgtgtgtctcccgaagacctcttctgatcacctcgtctcaggccaccatctaatgattaaggctacagatgctttttatgctaaggaaagaaagaagagaaagataaaaaaagacgcgccatcgctgtgcagggaggcaaaagaaaatagtggtgtgtggttagagaaagccggcttctcgaccacttcgtcaatgcttgtaattataaatcctctgtaggactgggtgtcaggtcaaagtgagaattctcgcgagtcggactgacgcattcgctcagcctccgctcaaaccactcgtttggtgggttgacagacgagttacgcgtagaaaagcggctctggcggtctgctctttccagagccagtctctcggccgcttgctccgctgccctggtcgcccgatccgaagttccagagaacgtctttttgtgcataggaaccatgggcttgtgtccattcacagaggaacttctgggtgctccgctgaggcgttttaccgttttcgatgtttgcgcggtctgtccgatggcttcctcttcgtcgccgggcaatgatggcaatggttttggtgctggtgttgcccaccgtctctttcttccgttattgaaggtccaaaatctgaagctgccaaggaagctgccatcggctcctggtacatgttgccatggcttttctctccagccgaagacaatgcaatccaccgtaccagcaaacgttacacagaaattcgcgagcagcgccaggataggaatcggatgtgcggcatacttgtcgctgtagttgaaagaatgataaacaaatggtatgatccagaccatgagataggtcactgggtagatgaagagaaggcggagttgtctttggatcgctcttcgtctcttcttgagaggcgtgtcggctgattccatgtctatcttgtctccgctcgtcaattgatgtccttcttcgattggtgcgagattgaccgaaggattcgcaacagcagtagtcatgtcagcagaagtcctccaggttgcaatggtagagacggactgtctttgctgctttgattctgttggcacttgcaggagttcctgttggatgcttccgtccgcaagaataatgacatgactgcttcgtctggagtggccaccggagggcagtttgaacccttcggaagaagagttgagagaggtcggccaggtggactgacttccctggctggagctgcttgcgggagcttggctgttggaaacattactgtctgtgaaacctgatagatcctgcagttcgcctgcgctagcacctctctttttgctcacccgcaggtcttgcggtccttctccgggcatggttccgaggctctctgccggtccgtagccgtttgtctcggtgcttgattccttgtcgtcctcgcgggcaaagatcttgaagccttgaccgacatgtaagtagatgcgtatggccacaaacatgatgtagagccagatgagatatcgagggatccattgcagtgctagtcggtaccagtaagggcgaatcggcaaggtgcaaaagggaccctgtgctatatacccttcacgtcctctgatgaaggcaagaacggcgcatagaaaaggaaacagaaaccatcccgccataacagtatgtctaaagcggtagagaccatcatggccaaagactttgctgccagtgcagaatatctgcaggtacatatgcagcgacatgatgaagattgcataatctgttcggcaagatcagcaccgcatcctgcaaagagaagtgcattgcaagactcaccacaagccatggttgacccttgcaacaagaagccgaatccttga

SEQ ID NO: 6 - DNA, 2866 bp, Aureobasidium pullulans
  source 1..2866: mol_type = genomic DNA
atgaaacgctctgaggtcagccaatgcgttccgcaatctccgacatgatgttatttgacccttgaaccatcacgagtcggcaacagtattggaacagattgaagtttgtgaaaagatgcatgaattcgccatgactttaaaatgaaataattactgaatgttccgtggtatctactgcgtctcttgaacctttcccgtaacgacacgcgatttgtaaacgaattttctaatgctcgagaagtctcaagctaccctggacaacaacgttctcaaggatggaacctggaggaatgtcgatggtctgaccctcgctggcaacaatgatgacggtacccttgaaggtgacgccacggccaaggttgacgggaccagtgatggtcaagtggtcgagctcgaggatcttgggaatgctggggatgcggctctggaagctcgagaccttcttgaagtcaccaccgagcttgatgaggggagcgggaccgaagcggttggggtccatgatgagctgaccgtgcttgagggtgtaaaggtcagacttgaccagcatcaagtcggagcaggtcttgacgggaaggaaacgacggcgggggacgttgacaccgtgtgcgttgttgaagtggcggatggcggcaccgacggcggtctcgagctggacgatgctgacatcggcctcgcccttcttgtcggcggggatcgacttgccgttggggataatctccatggcgagctcgttgttctcgacgatgcgcttgacggcgcggaggttcatccagatgttgttggtgttgaagtacttgaatttcttgatcgacttgaactcgttgacgtgctccttggggacctgggcgatttcaagaagacgggcctggccctcgtagtcaatgatggtaccacccttgacatcggccttggtcttgtcggtcaattccatgatgtactcagccttggtatcaaccatgtgctggaggatcttgaggtcgacgacagcgccaaggttgtcggcattggaaaggaagacaatctcgacaccgcgctcgagcagcttgtcgaggatgccggagttgtaaagcgactcgaaaacgtcaccgtgaccgggagggtaccagtcagagatctgagagttggccgacttgggagcggggaggagggagtccttgaggacacgggggtatctggactggttgaaggtcatgatgtcgatgttgtgaccctcgtacttcttgatgatgttggcggtgtctgcgtcggtgttgaacgagttcatcaagacgaaaggaacgttgacaccgtaggtgcggttgaggtactcaatctgtctaaccgaaaggtcgaggaaagacatgccgtcacggacctcgatgaccgacttggggccaacacagcccatggaggtaccgagaccaccgttgagcttgagaacggcgagcttgttcaagaactcgacggactcctgctgaggcaggtcctggtaggcaacgacctgctcgggcttgggaggagcaatgcgagaccactcgatctcgttgcccttggccttgtcgttgagatatctgcggaagagagcgaagaagttgtccatctcagtctcgaaacgctattcgtctgcgttagatgctgcaggcagaggcatggctatctgattgtagaagactcacctgcttctcagcggggtcagtgaccgagtcacacaggttgttcaaggcattgcgcatctgcgaggcagcgaccgaggtcgaggtgttctcgaaggcctagttgtacattgtcagcgagttgcaaaatcgagtcagagagctaaggagctggcggaagccggatttcctgactgcgccacggcacatgatgcagacagaagaatcaagcaggtgtgtggctaagcttgcaagcgtgagatcgaaaacgcatgtaacaaagtggggcccagaatgatcgtgtcagctcagctttgcgggaagcggatgatcgagcttgccaccgagtagcagcagtcttgggcttctctgcctcgaaacaatgacaccgtgtcacaaaataaggctcacgcccagccagctcgtagaggaggggtagtcaacgtacaacgtgggattgggtcttgccgtggtgtctcttctcgaactcagggccgccattgggcttcaggtgggttgccatttcggaggccattgtgaatgatgtgaaactgaattgaactgtcgagctcgagggttagaagctgctgagtgcaacacgaacgagcgatggtgggcaattgacgaaacttacaagaagaaggtcaactcagaaaaaggtggttaggggaaagaaagagagggaaggaggacggctgaaaccagagcgccaggatcgtcgactttataacctgccagctccgcctttgggtcggggcgacgacacaacaataaattattagcggaatcaatcacagccttgtcagaatcgggttacagagctacactaacgccaagtctacttttccccgccgcagcacgttgagagtcgagcttcaacaacttgagctacggcaaggggttgggagacctactattggaggacgaaaaagcacttcggcacccatctgttcgagacactgttctttgacgtaacttctgcatcggcataattgcaacggctgtatgtatttgttcagactgcttcctttgagatccaatggccgtttgatttcaacgttgtacacgacgaatgccaatgcagcacttgcaacacctgcaccactgcaaataaacactgctgccgtgttaagtaggcaaagctccacctaggtagctcaagaagttcacgaactgatccgaccgagattaaagtggaacatggggatgtcaaagcgctcagcgctgctatttga

SEQ ID NO: 7 - DNA, 10540 bp, Aureobasidium pullulans
  source 1..10540: mol_type = genomic DNA
atgcgtgggaagaccaatctcgtcaaagaagccatgcagttggtcgatgatggttgtgacttggtgcgacagatggccagtgtccatgtcgtcggcggaggaaggggctgccggcagcctgttagggaaagagcgaatgaccagaatgccaggtgtcggtatttgagagcatgtactgtaacagggtggcagcttctgcttgcacaatgtcacggcggtgctctggacgaacaactcgactcggatgctgcacgcctcgttcacgccagtatcggcagtctggagtagcgtgccgagagatgcaacgatctatcttttgcaactgcgcgccctgaggaatgctcctagatagttactgccctctatcactcttcagttggacaaaacgtcaatctagacaacaagataagacgagagttgcagcagccgtcggtctcaatccgcttgaccatccactgtacttttaacaaccagcccgagtctttgttgtttggtacgccgcacccgcgttgcccagcaaccacctggtacaacctccaccatccaacctcttgtcaagtccctcgccgaaccacagctgaatcctacatggctgtctgccgtcccttgctaacccatgcttgaaatcaattcgtgtcgccgtaacctactgatttgtccgatctccaagtgttacacgtcccgcgccttcgtccgaggtcaagtgctaagcttgtggccgtcgtcgtcgtcgtcgtcgtcgtcctcaagtggcctctggcatcccactcgtataccgatcttttccaaaggagaactttgcttggttagattgtaaaaccatcacagatacttagcttggacctcaagccgggcattcttgttctcgcctctcgtggagccccaaggcgaccctctagaaaatgacgcctcttcatgtcaggatgttcccgtcccagaaacagtcgcatatcttcgcaatatcttgagaaaccaagcgagtccgtcttcaagtcctctcgtcctagatggtctgaattacgcccttaagaggcctggccaagcccttggcttgtgaccgctcttttctgcttgactacctattcgaaaactccgacggcgttggaagcgagcggtgagactgcaaatcgtctctacagcagtcttacctcccgtatgcgctgaagtgtaaaatgcgcgtagctgcgagaacacacgacgattactagaaacacgacataattcaccgagaggcccaccccgtcggctctccctagtctgaagacgtttttgaacttttgggtgccttttggtcgcctatcgtcgccaatcagcatatgaatagacgaggcacgtacgacacaagtgcctgccggggaattcgtgatgaagaacctcctacgaaccaagaatactgcgcatctgcattatgggctcgaggcgacgagcctttcgagagtaacgaggcaagtcgtgagggacatgagcactggagcagacgcaaaagtcaggtcagtgactctggctgtttcctgcccaactccggtcataccatcactgctgcattccccgttgcactgcctgtcagatcatgctcgtctgagtttacccaccctgaagttcttttatctgcttgcttgctttcttgctcgttcgcttgctgtctgtcggccgtacgcgtcgttgagtattctcactcttactctagctctgaaagctacctatcgcgttgagcttggcaccctggcgatagtcagtcgttccaactggatcagcagattgtatgcattgcgtcggttgggaaagtatatccctctcccattgtcacatcatgacctacgatgtgcatccaagtctcatgcatggcagctagtcatatcctgctccacgagagtacgcgtatatgtctgggcaatcggggatgtgacgatgttgtcgatagggtgactctttgtgatactcaagagtaatccacacgccaagcaccccgtttgtatgatcaccaccggtggctaaatggtaagggccaatagaggactgtcgctacttttctccgacatctcacatactggttacacgttgctggtgagcactcctgttgcgtgtcgcctacctcaggccgtgcggtattttgtactcgtcacgacgtcgcattgtgcccgtttcttcattgagcacgttagtggacccttaggatgatctctgtcggtctgcatggagcatagacttgcagacttgcgacgtctatctcgattcctgccgaaggagaccttaacgccgcccagatacatcaaatcaccaccgggcggtgatcacctcacagtactgtctgttgatcaggtccggtccgcaaccaagcgcactgctagaggggtccccacaagcaccgggttcgggctcttcgaggaacatggtattctagtgtacagtgcggctggaaacgggcgattcgtccagagacagctcgagaagccaggacctgaatgcatcatttctaatactagaaggacgataacattttgacggtgatataccaccgctgtgcgatatcaaatgctcagccacatcgtaatctctccgtatagaaacatccatatcactcagttcgcagatcatgggactccatcacgcttgaaacaagcggtttatatgccaagatgttgaggtcgctagtagccttcggacgatcccccgtccttgtgtgtggcaggtgtatggtagctttgcaattcagttggcgactgcatgcatggaagtgtagagatttttgcacatcgatgcgattgaccgatgtttcgtcttatcaacggtcacctcaaccatctacagttatctaggatctgcccaggatctacccagtgcagctctaagatgatcaggctcgtggtctgtgcaagaggtatccactcaccctcacaactcttgctgtgtaagtcctactctactgcaacggcgctgaacgatggtcaagctatcaaaatgcacatccacttcttgacttgtaaattagagtcgagtcgaccaagacccggggaagtcatgactctcagtgggtctcaagttgattttgagaggtcctgtgcaatgctaccttgcgcacactgcattctgaacagacagacatttggtagttgcttgacgtgtgtacgtatgcaatcgaacactctgattgctgggatgtcggtggagtcatgtaccatgcagtctcatatcctagttcacacacaagccatgtgattgtcagttactctgtgctgctccactcttgacgtccggtttcatttcgtacgacggagttcttcatccctgtcatctctcgtccaacttctccataccgttgactgtcgacccacacgtcaccccggctgcttggtcgaccattgcaaagacataaagtctcctggaaaaaacatgtgaagctgcaaagcaggatttagcacagtagttccaagacaatagaatgtcgccagaaatggccgaatttcattttgagattcgataaatcgtatatgctgccctattgttttcctgcatgaagtgtatctaaactttgtctcgcgggttcgcatactgacggctttggcttggcatgcgcgttgattatgctgcgacgatcgcaggatatcaaggacgaccttttttcgcacgtaccagaatacggatcttcattttttctgtttggttgaccgatggtaagagcatcgacttcacgccattagccaacatcccctttcccccgatgtctgagtatgtgctatgacagcatttctcccaggtagcgaaaaaactctccgaaagagacttcgggacctcgcaccttatgcgtaaaaatcagagttatctgctgcaaaccacacaggtgcaactacaactaccgctttctaccggcggcgctgcaggctgagacagaagaactcacaaaacggcccttccgaccatgacgttgcagccatgcgtctgtgcctcaagacacatccccatcgatagcaatcacaaatctttaatcaatcgcatggtcgacatcggaataggcgcgacatgtcatatgtaccgtggcagtggtagccgcctagactacttctgattacgagactgggctatcgaatgttgaattagactcctttgtgtttctcagcctccactgtgcggcgcgagcagcaagatgcaggttctccctacttgttcgctagtgctcaaaccttcggcaaccagagcagctcgcgaaaattctacggccgttccgggtcaacccacgcgattcttgtctatcggctcgacatcgattggacccgaccggactcgactcgacttcgcatcgtcctcactccaaaattgcggctgcacctgcttttcagctcggctcagcccatacagcaacaagctgagttatcacgatttcccgttccccatgtctcgtgacaattttgacaaaccgatttaccccttcgccatctggccgccacaaagccctttcatcagctgcttccttgtcggggagcgatttatgtacggtaccttgcaggctagctcgcgggccaggccgcacggactacggctagccgctgagcttgaacagaaaaagatcttcgaccacggtcactaaattggcgttggacgtcaacaatccggcgcttccatgcttggttgaccaggatttctttcggctctcttgactctgccctcgggtttctgcagggatagagatgttgtgatagtcaagatcacgctgggatcggctcagtctgctcgctccaatgcactatgaaacctcgtcgtgcaaccctaaagcatgctgtgctattgactccaagccatcgatatcagctctgctagcctcacgtgcaaggccacgggggtgtcaatagagttaggcccaactcattgaggcacggacgtgcactcactccagatgctcaaaacggcttcgccgaggccatcgactatccaagctcggtaggcgtccgtccgtaggtctgcaattccaagtgctcaacatagctgcagactctgcagcatgcactagagggtggatgatttgattgatgcccagctgtctgatcaattgccctttctgagcgcacgttgaccatttgatctgcgcagctgtttagtctgtctggaactaggacccacgctctttacgcgttctagcacatcgttctcccttatgcttgttttccacatgttctccactaaagatagccgtgtctcaagcgtctctacgacctcagctgcagcctctgatcatgtcaagcgagtcacggacgtaaggtttgggttcactgacaaggcaccgttgaaatgggtggtgtaccgagccaacgggtctggtggtatcatcgggggattgttctactcatccctttatacatataggctcatcgaacgcctctcgtggctggcaattgtatccccttcctttccttaatctcgtcactatacaactttgtttgcttggagtctctgttgtccacacatatcattatcatccaccatggacaggactgcaattgccaactacgtctttcaagatgaactactagccacaagccaccatgaggctctttcaaattgcctgaacgctgccataaacaaccccttcatcaacaagaccaaccttctggcttcgctaccactatctgccgacactgccattgcatctttacggctactcacatcattgctcaacacattacccggatcctggccgacgtttgagctcacatgttgcttctcgagattgtggaaacttctacccgaagatgtgaaggctggcagagatacacagcacaacgccctcttggacgccttaacacaacagatgaagaacacctactcagatgtctgctctctacttcccgaatcagacaccccagcaatctatggtgcctcatcttcaaatcccatcagccacagaagactacatcagtttgttcgggaattcagtctacccaccaagcagactgctggaactagatcagtcgttgccttggctctaccaaatggtcctgtgctgggattggctcttctggctactgcaacatactacacagcagcccctgtgacggtagcttcaggaccggaacagtttcagagcgatatcatgcaagcaggagctagcatagtccttgttacacgaccagatgtccaaagactaggactcgagggctcttgggtccgcgatgctggcatcgaagtgatcatagtcgaacccaaggatgacgcaactttcaacatgacatatagcaacggctctcccgtggtcgtcagctcaaaatctacacctaatggatctgacgatatcgccctagtactattcacatcaggcacttctggtaagaagaaggtcgtcccttacaccgttcacaccatcatctccggtgtaggattcgtcatcgaatcctggggcttgaccaagaacgacatttgcatcaacatgatgcctctcttccatgttggcggcattgtcagaaatctgttcgctcccatcatggctggtggctccacaatctgctgcccagcatttgatgccaacttgttctgggacgtcgttgaggatctgcaaccaacttggtactacgcctccccttcaatgcactccgtcattctcgaagagggtcgtaaccgtccatcggcactggagaacagcaagatgagattgatttgcaacgccgctggtggtctgctaccttcgcttgcatgcacactacgagacacattcaaatgtatcgttcttccaagttacggtatgacggagtgtatgcccatctcgagtccacctctcgactatcaactcgatcgatccggtacatctggtgtcagtgttggtccagatatggccatccttgatggcaatgaccgcagacttcctattcacacagtcggccgcatcgctgtcagaggtgcacctgtgtttggtggatacctaaaagacaacgatgtgttggatacctcatgctttactgcagacggttggttcgacactggtgatatgggctatctggatgcagatggctacctttacgtcactggtagaagcaaggaggtcatcaacagaggtggtgaattgatctctcccttcgagatcgaggaagccgttgttactgctgctaagcgtgaaggcaaccctctcttcggtcgcgtctccgctgcccttgcattctcaatgcctcacgatgtgttgcaggaagttcccggtgtggtcttggttactcctcagggtgctcgtcgtgccgacatcaagacggttcaagaggccttgaaggactccttgagtcaagttaagtggcccgcgtacgttgtttacatggacgatcttcccaagaacaacaacaaggtgctgcgtatcaagctcgcagagcgtcttggcctgggtgagatcactgacgagacgcctttggcggatcgccatttcgaggcagcttgccctccacccaacactcctctcagcacccgcatcggtggctctatctgttctttcgacgcagcattcttgtcacgcaagctctcgactgttttcggcacggagattgacattgctgtgagaaaggcgtctcatgatggatacccagaagccttcctcgctcctcgcaagaactctactgtcaccccagtcatgtcaagctaccctagcttcgactcactcctccgcgctgagcttgacggctacctcatcccaagcaaagtgcactgcctcgatgttcccttgcctagagatagggagggctctgttgacgatgctcagctggagactctgatcgagaacgattctgatgagattggtggagacatgaccccaaccgaagccaagattgccgaagtcttcgttcgcattctttcatgcgctgcgtccgagcttactcctgaatctgacttcttcgctttgggaggcgacagtcttcgtgctggccgcttgctgtctgaacttcgtaaggagttcaaggttcgcctgcctatcgatctacttttcgtgcacagcaagatcaacgcattggccaagaacattgacggaatgcgtggtcctactgacgagaagcatgcttctgccgaagctgagcagactcctgtcaactacggctgcgaaaagacgtacagctccaccaaccctattgtcctcttgatccagctgtttcccatcggtttgctttatcccatgaagcgtgccttgacctggacatgtttcatgtacatgctcagtgccactcgtgactggatcactgcaactagactcatcggacgtcttttcaacctgatgctcgctcttggtattgcaaaggccgtcactctcatcgtctgtccacttcttgccatttgcctcaagtggatgataattggtaaatacaaggagggcatgtatcctatgtggggcccttaccacacgcgctggtgggtcacagacaagatcatcaccatcctcggaaaaggtttctttggttcatctgaattcggtatcgttctgtactaccgcttgatgggtgcaaaaattggcaagggtgtcaagattgccaaacgtgctaccattcgcgagttcgacctcgtcactctcgaagacggagcaaacttggaccagtgcatctgcagaggctttggtgccgagcagaacaccagcatgtaccttggtcatgttcgtgttggtaaggcagcaagtgtcggtcttgcgtctatcgtcgcccccggaacctcagtccccgacaatgcatgcattgggcctaactcatcgagctgggaagctgatgatgctagtgaagaattccgcgacttatccgccagcaagattcctggagctcaccctatcctgtccttcttgttgatcacccccatctctttgcttgtcactctttgcaagaacggtccctggatcggtggtctggtcggtcttgtgttgacgcagcccaagcccacttcagacaaggtccaatcgatcattttgtggtttgctgtgccccatcgtatcggctggcatttcgctgcaaagatttttggtgctggcgcaggtccgctcgcgtggttctttgccgtttgggtccttaagaagattttggatttgaccatcggtaaacttactccaggacccgctcagtcgagatcgcaagcccaacgcttccgtatggcggtgctcaaagccgttgcacctcccgctgcattccacaaagtcactgagcttttcggtggtcattacgaggcaacgtcagtcctttatcgtgccatgggcgccaaggttggacaacgcgtgtactggcctggaactggtccttcgttccaggacttcgacctcctcgatatcggaagcgacattgtctttggctctcgctctcatctcgtaacctccgatggcactggaagtgactatatccgtgttcaagatggcgccatgatctctgaccgtgtcgtgctcctgcctggcgtcactgtcggcaagaagagtgttcttggtagcggtgctttgactcgcagaaacaagtactacgagcaggagacggtttgggtcggaagcaaggctggcgaagctgtatgcctcagcggtaacaagcgctcgaccacctcggatgctgctaacactcaacatggcatgcccagcttgaacagcactaccaattctagcagcgtcacattgagtgaaaaggaaaaggccgactacgagaaagcagtccaagagggtgtgttgagcagcagctcatctatgactatggtcgagaaagaacaggcagctgggtcttcctcgcctttcggtcgcgccttctacgagggcaaagccccctaccacgtcttcggtccttggaccattttcctctactcgaccttcaccaccatctttgtcgctttctactggaacatttcttccatcggtgccctgcaacttgtcgccaagcttatcaagacatccgatcctgtacacaacccagagactccctaccgccctgttctcattttcgccctcttcgccgcctttatcgctgtgcttcacactgttcaggctgtcttcgctcttgccatcgtcgtcgctgctaagtggatccttcttggtagacgccaggtcggcaactacgactgggacaagagcagctactgccagcgttggcaattgttcctcaccgttgagaaacttcgtcgtcagtgctatggtggtgctggtgtcgtcggcctcctcaccggtactcacttcgctgtcatgtacttccgcgctcttggtgccaccatcggcaaggactgctccctcttctcgggtggtctgccttcgctgatgttcaccgagcctgatctccttactcttggcgaccgcgttgccgtcgacgatgcatctctggtttcccacatcaactctcgtggtgtcttcaacctcaaccccttggttgtcggcgaccgcagtgtgctcagaagtggttcgagattgctgtctggtgccaacatgggcgaagacactatgctgctcgaacacactctcgtcatggctggtgatgtcgtcgacgatggtgtcacttaccaaggatggcctgcagagggctttgagggccatcgcgcacctcttcgcaacggccctgtcaccatggtggctgaggcctaa

SEQ ID NO: 8 - DNA, 378 bp, Pichia pastoris
  source 1..378: mol_type = genomic DNA
cgacagttggctcatcatccgttacatcatctttttttctgctggctccgctcggtgggctcccaacgaagcagcaaaaaaagtgagagagaaaaactagcttggcggggcatccgaagctagaccctttggctcgcttagtcagtgcgccctctcacacacactcaaaaaggccacccctcccgcaccctcttctcatcaccgtcttcataccacggttcgtcaagcaatcgtatctggtaagctttgacctcttgagcgggctccactttgctatttcttggatctgctcattcttttcttccttcctctctttctaacctctcttcagaaagttcaaccgtacttcactcaatcttccatacatcaccgtcaaac

SEQ ID NO: 9 - DNA, 3781 bp, Aureobasidium pullulans
  source 1..3781: mol_type = genomic DNA
atgttctcgtgcgtcgtcgcacagagacacaaacattcaatgtgtcgaaaggtatgtatgggcgtcatccgcccctgtccaaggctcgatgtatgcgatggctgacttatcaagtacatatgatgttccagccaagccaggtttacgatgagacgcgtaccacacactcagcaatggcaggggcttcggacaccgcctcatcattgcattgcccttttgttgctcaacgcctccttggctcccctttttgcatcctagatttccaagacagctttccaagacagcggacttggtatgccagggcatggcagagaaacggacgtcaatatgccaagtcgcctcctttcgaattggcttttagcgcggagagctgaggaacgtgcatgttcacccaaatttggcttcttccacgagggacactcccctgtcagtacgaatacgtgacacaactggcttcgcggcggggatgagcggtctctagcatcagactgcaaactgcttgtcaaatggatccgaaactggccgctcgcaggattacgacagaccaataaatcgcagcagatttgcacagcctgccaatgtgattccttggcttacttggcgtataagctctggtttagatgccctacttcatggtaccattgctggaaagtggacaagtccgtgtcccgtgtaaagccaaggagaacgcagatgagtgaaaactggtggaccgtccgacagtcaagccatggttgaatgtttcagtcatgcccaacaacaagagaaaattcaaagttccaatggtgacgatatggtaacgatggctgtggtgggtgcgtatttaccaacactgcattagcgcttgcgaatcgggtaggaaggagcgaccggctcttggcggctttccaagtcttcccccgcgccgagaacgagtggaagtcgctcttgtcctcttaatacttgagcctgcgcccgctgcttcccttggctctcacttctctacacggtcttgaccttccttccgccttcccaagactttttacatttcacgtgttggacacctctttccttctgcacgtgcaaaaccagattcacatacttatacaggcttctgtctgtttgtgttgcatcgcgcatttcgacgcgttttttccagagcgtcgctgccaagtaccaagaccatccaaagtccgaacaccaagatgtctgatgctgcacctggtgccgctatggcagctcagccaaaccaccctgaccctgctgccgctgccgctgccgccgctgctgctcaacaactccaacaagcatcgcctgatcagcaacatcagcagttgcaaaattcactccagcagcaccagcctctgcagcaacagaccctagcccagcagtcgcttcaacagtctcccactatccaacatgcccttcagtcgcccatgcagcagcatgcctcgcctcaacctgttcagcacgcccttggaaccccttctctcggccaaagcaccactgtcgactctctcacttgccagtggctgaactgtggtgaacgtcaccaaacagctgaggctctctacgtaagttctcttccaccttacagcatgcccattatctaacacctgcattaggagcacgtatgcgaacgtcacgtcggtcgcaagagcacaaacaacctcaacctgacttgccattggggttcttgccgcgtcacaaccgtcaagcgcgatcacatcacctctcacattcgcgtccacgttcctctcaagccccacaagtgtgacttctgtggcaaggctttcaagagacctcaggatctcaagaagcacgtcaaaactcacgccgatgactcggtccttcttcgttcgcctgatccgaacatgggccagaacagaggccctggcaacaactaccaacaagcacaacgtaagtccttcaactttggttttgccgagtatgtatgctaacattgtctccagcttccaacggctactacggtcatgacttgggagcctccagcggttaccctcaccccagccacggcttgaacaacaacaacttcttccagcagcctcagcagcagccccagtatgccggctatggccaggtcaactaccctgccagcaacagccttgccgacattcagagtatggacactcgccgccgcgccatcgaagctcttaacgacttcctcggtgacattaagcgtcgcgctatcgaccctggcaactactacgatgttggccagcgtctccagtcggccaacctgccgctccccatcagcaccggctacggctactcgactggcaacaactattccagcaacaacaactcgtcatataacactgccagctcgttgcttgacagcttcaacaactctggcaacttgggcatgactagcaacaacggtggcagcatgtctcagggtcctcttacccagtcatactcgctgcctctgccctcgaacgctcgtaccaagcaggatctggttgagattgataagttcctcgagcagcttcaagctaccgtctacgagagcagcaacagtgccgctgccgccggagtccagcagcctggatcgcatgctcagtacaccaactaccccagcttcggcagccagtaccgtagcagcgacagccctcctagcatgcaacactctggctctgtctctactcctggagttggtcctctctctaacatcaactctggcagtgccatggagactcctgccctcactcccgcttccgtgtcatcatatacctcagctggccaatctcccatgtctacccactctcgctctagcatgagcagcgctcacggcagctccatgtaccccagccttccatctgtcacagccatctctgacatgggcgctggttaccctgcaaccacttctgctcctgcatccgggcttgcctctggattcgagggcttcgagggtcgtcgctacagtggtggtcgtcttcagcgcgaggctcctgctcctcgctctgcagacatggattccgatgccatggagactgagtctgatggcaacaagactcctcgtgccaagcagcccgaagactcgtcttctcttgaccccgctctccgtgctgcctctcaatcatcgactgccgtccaaagccccagcactggtgcaggcagtgattccgacgagaagcaagccacgtgggtcgagaacgtcagagttatcgaggctctgcgcaggtgggtcagtgagagaatcaacaaccaagagttcgagaccgatcccgagtatcccgaacctcaacaccacgttgatcacgatatgcacatggatcaccaccagcctgctgatgagaagactctcgagccccagtacgagcagcttcatcaccaagagcacgagcagcagcatgagcagcaacacgagcaccagcctgaacaccacgaacaacacgtacaacagcatgagcaacaacagcccgcacctgaaggtgaagttgatgtttcctaccctgttctgaaggttgaagagtaagggagttgtccctattcttttcctgtggcgtgtacggtatgttactattctatatggcagatcacgagtaaatggaacggatcagattgaaggcgttgggctcgctcgctcttaaaagacgagcttgaatgggcgttggtgatggaaatggcgggacaaggtggctgttctttgcagcaatgtggtttccctgctttcagtatttctattttggtcaaggactcaggcagtcggcatttacataccccgtcgctgtttgcatgttctcctattggcaactctaatttcattacattactttga

SEQ ID NO: 10 - protein, 603 aa, Aureobasidium pullulans
TTLVTGIAIARATSNSNNTINDDKLHTWWHNSGEITRSAVQPASVRQSDLYSIKVTNSVDQTYYDSFVYQTIPRNGQGNILIPNDPSSTTTASDGITIEEAIGMTMSWTSFLYSADVWVKVHRLDNFSIEADSFVIRPTNLNLTTSVANGDLFIQVPYNGQSRKFSVEFNDNLYEFHDGCSSPSCSYVQDTNASGPYYVDAYDDSMPLMAVEPLDSLFIFASPFEDESLVPDETADNVLIVEEGLISGLNTTQANTVIFKPGVYYATATDYLNLSATVDWLYFAPGAYVKGAVEYHTTSALIKATGHGVLSGEQYVYQADPTNAFQNHNVNGSPLRMWKGTVPYGQQTTWLVNGPTLNSPPFNSMDWYGDMSSLSVLCTDYKQVGGFFGQTDGMEAYPGSVYQDIFYHTNDDTIKVYYSDVTISNVLVQKATTAPIIQFGWASRNLSNIQVENISLIHSRWNSNGSNPGLIGSNNVYDPSTSSTAAMNTSTADTYTTAQDITFSNIRAEGISGPLMRIYALENLSNITISDVWIEKFGSCDGYENIGISESFMPMMTDENGNNVTVDGFVISNFMVGDEKVTLDTASSVGQLYWDATFDVTIE

SEQ ID NO: 11 - DNA, 2412 bp, Aureobasidium melanogenum
  source 1..2412: mol_type = genomic DNA
atggctgaaactgaatctttgtcacctttcggtaacgctagagctactgttactgatcaaccactatccaaggacgaagtcgataagatcaacagatactttcacgcctccatgtatttgtgtttgggtatgttgtatttgagagaaaacccactattgcaagaaccattgaaggttgaacatatcaagcctagattattgggtcattggggttcagatgccggtcaatcttttacttacatccacatgaacagattgattaaaaagtacgatttggatgcttttttcgtttctggtccaggtcacggtgctccagccgtcatttctaactcctacttggaaggtgtttactccgaagtttacccagataagtcccaagacgctgagggtatgcaaagatttttcaagcaattctccttccctggcggtattggttcccacgctactccagaaactccaggttccattcatgagggcggtgaattgggttactctatttctcacgctttcggtgccgtcttcgataacccagacttaattgctttaactatggttggtgatggtgaagctgaaactggtccattagctacctcttggcattcaaccaagttcttgaatccaatctgtgacggtgctgtcttgccagtcttgcacttgaacggttacaagattaataacccaaccgtcttgtccagaatttcacacgaggaactaaaggctttgttcgttggttacggttgggaaccatacttcgttgaaggttctgatttagactctatgcaccaagctatggccgctaccatggaaagagccgttaccgaaatcaaagctatccaaaaaaaggctcgtgattctggtaaggccgaaagaccaagatggccaatgatcgtcttaagaactccaaagggttggaccggtcctagaaccgttgatggtcacttcttggaaggttactggagagcccaccaaattccattaacaaacgttttgaaggataacgaacaattaaccttgttagaaaagtggatgagatcatacgaaccagagaagttgttcaaaaacggtaagttcattgacgaactaagagacatcgctccatccggcaacagacgtatgtctgctaacccagttactaacggcggttctttaagagctaacctaaaaatcccagactttaaaaagtttgctcaaaaggttaataagccagccgctgaacaagctccatccatgaacctatttgccgaattcttgactcaaattgttaaggctaacccaaataactttagattgttcggtccagacgaaacccaatccaacaagttgggtgctgtttacgatgttaccaagaaagtttggatggctgactactttgaggaagacaaggatgacggtaacttggctccatctggtagagtcatggaaatcttgtctgaacacaccgttgaaggttggctagaaggttatttattgactggtagacacggtttattgaactcttacgaaccattcatccacatcattgactcaatggttaaccaacacgccaagtggttggaaaagtgttctgaagtttcctggagacaagaagttgcttctttaaacatcttattgacttcaaccgtttggagacaagatcataacggtttcactcaccaagacccaggtttcttggacgttgtcgccaataagtctccagaagtcgttcgtatttatttgcctccagacgctaactgtttattgtctaccatgaatcattgtttaaagtcctcaaactatgttaacgttattgttgctgacaagcaaaaccatttgcaatatttgaatatggaggacgctgttgtccactgttctaagggtctaggtatttggcaatgggcctccaactctgagcaccaagaaccagatattgttatggcctcttgtggtgacgtctctactcaagaggctctagctgccactgctttattgagagaatacttaccagaaattaaggttagattcgttaatgtcgttgacttgttcaagctacaaccacatggttatcacccacacggtttgaaagatgacgaatacgaagctatttttacctccaacagaccaattgtcttcaactttcactcttacccatatttggttcacagaatgacctacaaaagaaaaggtcaacacctattgagagttcgtggttacaaggaaaagggcaacattgacaccccactagaactagctattagaaacgaaaccgacagatattctttggctatctgtgctatcgactctttgaagtccgttttgggtaacaagggtgcttccaccagagaatacttattgaacaagcgtactaccgctatgaattacgcctacgaaacaggtcaagatccagatgacttcgttaattggacatggaactac

SEQ ID NO: 12 - DNA, 2439 bp, Leuconostoc mesenteroides
  source 1..2439: mol_type = genomic DNA
atggctgatttcgattctaaggagtacttggaactagttgataaatggtggagagccactaactacttatccgctggtatgatcttcttgaagtctaatccactattctctgtcactaacactccaatcaaggctgaagatgtcaaggttaagccaatcggtcactggggtacaatttccggtcaaacttttttgtacgctcacgccaacagattaattaacaagtacggtttgaacatgttttatgttggcggtccaggtcatggtggccaagtcatggtcactaacgcttacttggacggtgcttacaccgaagactacccagaaatcacacaagacatcgaaggtatgtctcacttgttcaagagattctcattccctggtggcatcggttctcatatgactgctcaaactcctggttcattgcatgaaggcggtgaattgggttactctttgtctcatgctttcggcgccgttttggataacccagatcaagttgcttttgctgtcgttggtgatggtgaagccgaaactggtccatctatggcttcctggcattccatcaagttcttgaacgctaaaaacgatggtgctgttctaccagtcctagacttgaacggtttcaagatttctaatccaactattttctctagaatgtctgacgaagagattactaagtttttcgaaggtttgggttattctccacgtttcattgaaaatgacgatatccacgattacgctacttaccaccaattggccgctaacattttggaccaagctattgaggacattcaagccattcaaaacgacgctagagaaaacggtaagtaccaagatggtgaaattcctgcttggccagttatcattgctagattgccaaagggttggggcggtccaacccatgacgcttctaataacccaattgaaaactctttcagagcccaccaagtcccattgcctttggaacaacacgacttggctactttgccagaattcgaagactggatgaactcttacaagccagaggaattgttcaatgctgacggttctttgaaagacgaattgaaggccattgctccaaagggtgataagagaatgtctgctaacccaatcactaatggcggtgctgatagatccgacttgaagttgccaaactggagagaattcgctaacgatattaacgatgacactcgtggtaaagagtttgctgattctaagcgtaacatggacatggccactttatctaactatttgggtgccgtttcacaattgaacccaacaagatttcgttttttcggtccagacgaaactatgtccaacagattgtggggtttgttcaacgttaccccaagacaatggatggaggaaattaaggaaccacaagaccaattattgtctccaactggtagaattatcgactctcaattgtctgaacaccaagccgaaggttggttagaaggttataccttgactggtagagtcggtattttcgcttcttatgaatctttcttgcgtgtcgttgacactatggttactcaacacttcaagtggctacgtcacgcctccgaacaagcttggagaaatgactacccatctctaaacttaattgctacctctactgccttccaacaagaccacaacggctacactcatcaagacccaggtatgttgacccacttagctgaaaaaaagtccaactttattagagaatacttgccagctgatggtaactctctattagctgtccaagaaagagctttctccgaaagacacaaagttaatttgctaatcgcttctaagcaacctagacaacaatggttcactgtcgaggaagctgaagtcttggctaacgaaggtttgaagatcattgattgggcttcaactgccccatcctctgacgttgacatcaccttcgcttccgctggtactgaacctactattgaaaccttggccgctttgtggttaattaaccaagctttccctgatgtcaagtttagatatgttaacgtcgttgaattattgagactacaaaagaaatccgaaccaaacatgaacgatgaaagagaattatctgccgaggaattcaataaatacttccaagccgatactccagtcatcttcggtttccacgcttacgaaaacttaattgaatcttttttctttgaacgtaagtttactggtgacgtttacgtccacggctacagagaagatggtgatattacaaccacttacgatatgagagtttactctcacttggacagatttcaccaagctaaggaagccgctgaaattttatctgctaatggtaaaattgatcaagccgctgccgataccttcatcgctaaaatggacgataccttggccaaacacttccaagttaccagaaacgaaggtagagatattgaggaatttaccgattggacctggtctccattgaag

SEQ ID NO: 13 - DNA, 999 bp, Clostridium kluyveri
  source 1..999: mol_type = genomic DNA
atgaagttgatggagaacatctttggtttagctaaggctgacaagaaaaagatcgttttggctgagggtgaagaggaaagaaacatccgtgcttccgaggaaattatcagagatggtattgccgacatcattttggtcggttctgaatctgtcatcaaggaaaacgccgctaagtttggtgttaacctagctggtgtcgagattgttgatcctgaaacctcctcaaagactgctggttacgctaacgctttctacgaaattagaaagaacaagggtgtcactttggaaaaggccgacaagatcgttagagatccaatttacttcgctactatgatggtcaagttaggtgatgctgacggtttagtttctggtgccattcacaccactggtgatttgctaagacctggtttacaaatcgtcaagactgtccctggtgcctccgttgtctcttccgttttcttgatgtctgttccagattgtgagtatggtgaagacggtttcttgttatttgctgactgtgctgtcaacgtctgccctactgctgaagagttatcttccatcgctatcaccactgccgaaactgccaaaaacttgtgtaaaatcgaaccaagagttgctatgttatctttctccaccatgggttcagcttctcatgaactagttgacaaggtcactaaggctactaagttggccaaggaagctagacctgacttggacattgacggcgaattgcaattggatgcttcattggttaaaaaggtcgctgatttaaaagccccaggttccaaggtcgccggtaaggctaatgttttaatcttcccagatatccaagctggtaacatcggttataaattggttcaaagattcgctaaagccgaagctattggtccaatttgtcaaggtttcgctaagccaatcaacgatttgtccagaggttgttctgttgacgatatcgttaaggtcgttgctgtcactgctgttcaagcccaagctcaaggt

SEQ ID NO: 14 - DNA, 969 bp, Aureobasidium melanogenum
  source 1..969: mol_type = genomic DNA
atggctgatttgttctctactgttcaagaaaaggtcgctggtaaggacgtcaaaatcgtcttcccagaaggtttggatgaacgtatcttggaagctgtttctaagttggctggtaataaggtcttgaacccaattgtcatcggtaacgaaaacgaaatccaagccaaggccaaggaattgaacttgaccttgggcggtgttaagatttatgatccacacacttacgaaggtatggaagatttagtccaagccttcgttgaacgtagaaagggcaaggctactgaggaacaagccagaaaggccttattggatgaaaactacttcggtactatgttggtctacaagggtttggccgacggtttggtttctggtgctgcccactctactgctgacactgttagaccagctttacaaatcattaaaaccaaggaaggtgtcaagaaaacttctggtgttttcatcatggctagaggtgaggaacaatacgtcttcgctgattgtgctatcaatattgctccagactctcaagatttggctgaaattgctattgaatctgctaacaccgctaagatgttcgatattgaaccaagagtcgccatgttgtcattctctaccaagggttctgctaaatctgacgaaaccgagaaggtcgctgacgctgttaagatcgctaaagagaaggccccagaattgactttggatggtgaattccaattcgatgccgcttttgttccatctgtcgctgaaaaaaaggcccctgactccgaaatcaaaggtgacgccaacgtcttcgttttcccatctttggaagctggcaacatcggctataagattgctcaacgtttgggtaactttgaagctgtcggtccaatcctacaaggcttaaacatgccagtcaacgacttgtctagaggttgcaatgctgaagatgtctacaacttggctttgatcactgccgctcaagcttta